SEQUENCE LISTING (2 sequences)

SEQ ID NO: 1 - RNA, 50 nt, unidentified
  source 1..50: mol_type = unassigned RNA
  modified_base 4: mod_base = OTHER; mod_base = OTHER; note = uracil
  modified_base 11: mod_base = OTHER; mod_base = OTHER; note = uracil
  modified_base 12: mod_base = OTHER; note = uracil
  modified_base 15: mod_base = OTHER; note = uracil
  modified_base 16: mod_base = OTHER; mod_base = OTHER; note = uracil
  modified_base 24: mod_base = OTHER; mod_base = OTHER; note = uracil
  modified_base 28: mod_base = OTHER; mod_base = OTHER; note = uracil
  modified_base 30: mod_base = OTHER; mod_base = OTHER; note = uracil
  modified_base 31: mod_base = OTHER; mod_base = OTHER; note = uracil
  modified_base 32: mod_base = OTHER; mod_base = OTHER; note = uracil
  modified_base 36: mod_base = OTHER; mod_base = OTHER; note = uracil
  modified_base 37: mod_base = OTHER; mod_base = OTHER; note = uracil
  modified_base 38: mod_base = OTHER; mod_base = OTHER; note = uracil
  modified_base 40: mod_base = OTHER; mod_base = OTHER; note = uracil
  modified_base 42: mod_base = OTHER; mod_base = OTHER; note = uracil
  modified_base 43: mod_base = OTHER; mod_base = OTHER; note = uracil
  modified_base 46: mod_base = OTHER; mod_base = OTHER; note = uracil
aaatcaacccttacttgcggcgctcggtctttacgtttctgttagtgccc

SEQ ID NO: 2 - RNA, 221 nt, unidentified
  source 1..221: mol_type = unassigned RNA
  modified_base 5: mod_base = OTHER; mod_base = OTHER; note = uracil
  modified_base 8: mod_base = OTHER; mod_base = OTHER; note = uracil
  modified_base 14: mod_base = OTHER; mod_base = OTHER; note = uracil
  modified_base 18: mod_base = OTHER; mod_base = OTHER; note = uracil
  modified_base 20: mod_base = OTHER; mod_base = OTHER; note = uracil
  modified_base 34: mod_base = OTHER; mod_base = OTHER; note = uracil
  modified_base 38: mod_base = OTHER; mod_base = OTHER; note = uracil
  modified_base 36: mod_base = OTHER; mod_base = OTHER; note = uracil
  modified_base 39: mod_base = OTHER; mod_base = OTHER; note = uracil
  modified_base 40: mod_base = OTHER; mod_base = OTHER; note = uracil
  modified_base 42: mod_base = OTHER; mod_base = OTHER; note = uracil
  modified_base 42: mod_base = OTHER; mod_base = OTHER; note = 42
  modified_base 43: mod_base = OTHER; mod_base = OTHER; note = uracil
  modified_base 45: mod_base = OTHER; mod_base = OTHER; note = uracil
  modified_base 53: mod_base = OTHER; mod_base = OTHER; note = uracil
  modified_base 58: mod_base = OTHER; mod_base = OTHER; note = uracil
  modified_base 60: mod_base = OTHER; mod_base = OTHER; note = uracil
  modified_base 71: mod_base = OTHER; mod_base = OTHER; note = uracil
  modified_base 73: mod_base = OTHER; mod_base = OTHER; note = uracil
  modified_base 83: mod_base = OTHER; mod_base = OTHER; note = uracil
  modified_base 95: mod_base = OTHER; mod_base = OTHER; note = uracil
  modified_base 96: mod_base = OTHER; mod_base = OTHER; note = uracil
  modified_base 97: mod_base = OTHER; mod_base = OTHER; note = uracil
  modified_base 98: mod_base = OTHER; mod_base = OTHER; note = uracil
  modified_base 101: mod_base = OTHER; mod_base = OTHER; note = uracil
  modified_base 105: mod_base = OTHER; mod_base = OTHER; note = uracil
  modified_base 108: mod_base = OTHER; mod_base = OTHER; note = uracil
  modified_base 111: mod_base = OTHER; mod_base = OTHER; note = uracil
  modified_base 119: mod_base = OTHER; mod_base = OTHER; note = uracil
  modified_base 121: mod_base = OTHER; mod_base = OTHER; note = uracil
  modified_base 126: mod_base = OTHER; mod_base = OTHER; note = uracil
  modified_base 127: mod_base = OTHER; mod_base = OTHER; note = uracil
  modified_base 131: mod_base = OTHER; mod_base = OTHER; note = uracil
  modified_base 132: mod_base = OTHER; mod_base = OTHER; note = uracil
  modified_base 133: mod_base = OTHER; mod_base = OTHER; note = uracil
  modified_base 141: mod_base = OTHER; mod_base = OTHER; note = uracil
  modified_base 142: mod_base = OTHER; mod_base = OTHER; note = uracil
  modified_base 147: mod_base = OTHER; mod_base = OTHER; note = uracil
  modified_base 151: mod_base = OTHER; mod_base = OTHER; note = uracil
  modified_base 157: mod_base = OTHER; mod_base = OTHER; note = uracil
  modified_base 159: mod_base = OTHER; mod_base = OTHER; note = uracil
  modified_base 160: mod_base = OTHER; mod_base = OTHER; note = uracil
  modified_base 161: mod_base = OTHER; mod_base = OTHER; note = uracil
  modified_base 162: mod_base = OTHER; mod_base = OTHER; note = uracil
  modified_base 163: mod_base = OTHER; mod_base = OTHER; note = uracil
  modified_base 164: mod_base = OTHER; mod_base = OTHER; note = uracil
  modified_base 166: mod_base = OTHER; mod_base = OTHER; note = uracil
  modified_base 170: mod_base = OTHER; mod_base = OTHER; note = uracil
  modified_base 172: mod_base = OTHER; mod_base = OTHER; note = uracil
  modified_base 173: mod_base = OTHER; mod_base = OTHER; note = uracil
  modified_base 199: mod_base = OTHER; mod_base = OTHER; note = uracil
  modified_base 201: mod_base = OTHER; mod_base = OTHER; note = uracil
  modified_base 204: mod_base = OTHER; mod_base = OTHER; note = uracil
  modified_base 188: mod_base = OTHER; mod_base = OTHER; note = uracil
  modified_base 182: mod_base = OTHER; mod_base = OTHER; note = uracil
  modified_base 183: mod_base = OTHER; mod_base = OTHER; note = uracil
  modified_base 207: mod_base = OTHER; mod_base = OTHER; note = uracil
  modified_base 214: mod_base = OTHER; mod_base = OTHER; note = uracil
  modified_base 215: mod_base = OTHER; mod_base = OTHER; note = uracil
  modified_base 220: mod_base = OTHER; mod_base = OTHER; note = uracil
gcagtaatacgactcactataggacggcaacggtctatttgttctgcaggagtcaaatatgacacagcagatatacaggaaatccaaaaaagcgttttagtggctactaatcacgaaatctacccttaactttacaagaattacaatgcctcagggtcttttttatcagtctttaattgtattaccctgacccgccgatctactggtggaaccttcggata

(19) United States
(12) Patent Application Publication
(10) Pub. No.: US 2026/0174694 A1
(43) Pub. Date: Jun. 25, 2026

(54) Lipid Nanoparticle Formulations for Anti-Sense Oligonucleotide Delivery

(71) Applicant: NanoVation Therapeutics Inc., Vancouver (CA)

(72) Inventors: Jayesh Kulkarni, Vancouver (CA); Dominik Witzigmann, Vancouver (CA); Kevin An, Vancouver (CA); Daniel Kurek, Vancouver (CA)

(73) Assignee: NanoVation Therapeutics Inc., Vancouver, BC (CA)

(21) Appl. No.: 19/124,321

(22) PCT Filed: Oct. 25, 2023
(86) PCT No.: PCT/CA2023/051416

§ 371 Date: Apr. 25, 2025

Related U.S. Application Data
(60) Provisional application No. 63/419,005, filed on Oct. 25, 2022.

(51) Int. Cl. A61K 9/1272; A61K 9/00; A61K 9/127; A61K 9/51; A61K 47/24; A61K 47/69

(52) U.S. Cl. CPC A61K 9/1272; A61K 9/0019; A61K 9/127; A61K 9/5123; A61K 47/24; A61K 47/6911

(57) ABSTRACT

Provided herein is a lipid nanoparticle comprising an encapsulated oligonucleotide molecule, wherein the oligonucleotide molecule is single-stranded or double-stranded and has a length of between 5 and 500 nucleotides; and 20 to 70 mol % of a neutral lipid content relative to total lipid present in the lipid nanoparticle, an ionizable lipid; a sterol; and optionally a hydrophilic polymer-lipid conjugate.

### TECHNICAL FIELD

The present disclosure relates to lipid nanoparticle formulations for the delivery of oligonucleotide cargo.

### BACKGROUND

Lipid nanoparticle (LNP) formulations represent a revolution in the field of nucleic acid delivery. An early example of a lipid nanoparticle product approved for clinical use is Onpattro™ developed by Alnylam. Onpattro™ is a lipid nanoparticle-based short interfering RNA (siRNA) drug for the treatment of polyneuropathies induced by hereditary transthyretin amyloidosis. The success of this LNP delivery system paved the way for the clinical development of the leading LNP-based COVID-19 mRNA vaccines.

The Onpattro™ LNP formulation consists of four main lipid components, namely: ionizable amino lipid (DLin-MC3-DMA or “MC3” (dilinoleyl-methyl-4-dimethylaminobutyrate)), distearoylphosphatidylcholine (DSPC), cholesterol, and polyethylene glycol conjugated lipids (PEG-lipids) at respective molar amounts of 50/10/38.5/1.5. Onpattro™ is still considered the gold standard for comparison in studies of LNP-mediated efficacy and current approaches to the design of LNPs for use in the clinic make few deviations from the four-component system.

Of these four components, the ionizable lipid makes up the bulk of the Onpattro™ formulation and is present at 50 mol %. The ionizable lipid is considered vital for the in vitro and in vivo activity of the LNP system and therefore most work in the field has focused on improving this lipid component. The ionizable lipid, which is typically an amino lipid, has been carefully designed so that it is charged at low pH and near neutral at physiological pH. This allows for electrostatic interactions between the lipid and the negatively charged nucleic acid during initial formulation. Since the ionizable lipid is near neutral at physiological pH, toxicity and renal clearance is reduced. After cellular uptake by endocytosis, the acidic environment of the endosome leads to an increase in the net positive charge of the ionizable amino lipids, which promotes fusion with the anionic lipids of the endosomal membrane and subsequent membrane destabilization and release of the nucleic acid-based therapeutics into the cytoplasm to exert their effects.

With respect to the remaining three lipid components, the PEG-lipid is well known for improving circulation longevity of the LNP and cholesterol functions to stabilize the particle. Generally, however, comparatively less attention has been devoted to studying neutral lipids beyond their structural role.

While strides have been made in LNP-mediated siRNA delivery, it is widely known that the Onpattro™ four-component LNP formulation largely accumulates in liver (hepatic) tissues. The ability of LNPs to accumulate in organs and tissues beyond the liver would greatly expand the clinical utility of these delivery systems.

Studies have found that DSPC and cholesterol contribute to the stable encapsulation of siRNA in LNPs (Kulkarni et al., 2019, Nanoscale, 11:21733-21739). Despite these findings, subsequent in vivo studies by another group failed to show any clear benefit resulting from adjusting the levels of DSPC in LNPs to improve the extra-hepatic delivery of siRNA. These studies examined extrahepatic siRNA gene silencing in vivo with Onpattro™-type LNPs (MC3/Chol/DSPC/PEG-DMP) incorporating DSPC at 10 and 40 mol % (Ordobadi, 2019, “Lipid Nanoparticles for Delivery of Bioactive Molecules”, A Thesis Submitted in Partial Fulfillment of the Requirements for the Degree of Doctor of Philosophy, The University of British Columbia). It was shown that the 10 mol % DSPC Onpattro™ formulation had similar liver accumulation and blood circulation lifetimes as 40 mol % DSPC formulations (MC3/Chol/DSPC/PEG-DMG; 18.5/40/40/1.5 mol %). Further, the 40 mol % DSPC siRNA-containing LNP (siRNA-LNP) only performed comparably to 10 mol % DSPC formulations in bone marrow gene silencing.

There is thus a need in the art to improve the in vivo delivery of nucleic acid such as RNA using a lipid nanoparticle.

### SUMMARY

In certain advantageous embodiments, the disclosure provides a lipid nanoparticle (LNP) encapsulating oligonucleotide (herein “oligo-LNP”). Such lipid components include an ionizable lipid, a neutral lipid, such as a phospholipid, a sterol (e.g., cholesterol) and optionally a hydrophilic polymer-lipid conjugate. In particular, the neutral lipid is present at a content that is higher than that of conventional LNPs, such as at least 20 mol %, at least 30 mol % or at least 40 mol % (relative to total lipid content of the LNP). Such novel oligo-LNP compositions exhibit surprising improvements in biodistribution in tissues and/or organs beyond the liver. The ability of the inventive LNPs to accumulate in extrahepatic organs and tissues greatly expands the clinical utility of the delivery systems.

In addition, the inventors have found that, in certain examples of the disclosure, significant improvements in biodistribution of oligonucleotides, such siRNA LNPs, can be achieved by adjusting the molar charge ratio of nitrogen-to-phosphate (N/P) or weight nucleic acid/micromole of total lipid of the LNPs herein. In some embodiments, the N/P of the lipid nanoparticle may be 4-15 or 4-10.

According to one aspect of the disclosure, there is provided a lipid nanoparticle comprising: (i) an oligonucleotide molecule, wherein the oligonucleotide molecule is single-stranded or double-stranded and has a length of between 5 and 500 nucleotides; (ii) a neutral lipid content of from 30 mol % to 70 mol %; (iii) an ionizable lipid content of from 5 mol % to 50 mol %; (iv) a sterol selected from cholesterol or a derivative thereof, and (v) optionally a hydrophilic polymer-lipid conjugate that is present at a lipid content of 0.5 mol % to 5 mol %, wherein each lipid content is relative to a total lipid content of the lipid nanoparticle, optionally wherein the phosphatidylcholine lipid content is not egg phosphatidylcholine (EPC).

According to a further aspect of the disclosure, there is provided a lipid nanoparticle comprising an encapsulated oligonucleotide molecule, wherein the oligonucleotide molecule is single-stranded or double-stranded and has a length of between 5 and 500 nucleotides; and 20 to 70 mol % of a neutral lipid content relative to total lipid present in the lipid nanoparticle, an ionizable lipid; a sterol; and optionally a hydrophilic polymer-lipid conjugate, the lipid nanoparticle exhibiting at least a 10% increase in biodistribution in the spleen, bone marrow, heart, lungs, kidney, abdominal skin, back skin and/or ear as compared to a baseline Onpattro-type formulation of MC3/DSPC/cholesterol/PEG-lipid at 50/10/38.5/1.5, mol:mol encapsulating the oligonucleotide molecule, but otherwise measured under an identical set of conditions, and wherein the biodistribution is quantified in an animal model by detection of labelled lipid at 24 hours post-administration, wherein the neutral content includes no more than three different phosphatidylcholine lipids.

In another aspect of the disclosure, there is provided a lipid nanoparticle comprising: (i) an oligonucleotide molecule, wherein the oligonucleotide molecule is single-stranded or double stranded and has a length of between 5 and 500 nucleotides; (ii) a neutral lipid content of from 30 mol % to 70 mol %; (iii) an ionizable lipid content of from 5 mol % to 50 mol %; (iv) a sterol selected from cholesterol or a derivative thereof; and (v) a hydrophilic polymer-lipid conjugate that is present at a lipid content of 0 mol % to 5 mol %, wherein each lipid content is relative to a total lipid content of the lipid nanoparticle, and wherein the lipid nanoparticle comprises a non-homogeneous core, the core being surrounded at least partially by an aqueous portion, and wherein the aqueous portion is surrounded by an external bilayer, as visualized by cryo-EM.

In one embodiment of any one of the foregoing aspects, the neutral lipid is a phosphatidylcholine lipid that is distearoylphosphatidylcholine (DSPC), dioleoylphosphatidylcholine (DOPC), 1-palmitoyl-2-oleoyl-phosphatidylcholine (POPC), dimyristoyl-phosphatidylcholine (DMPC) or dipalmitoyl-phosphatidylcholine (DPPC). In another embodiment, the phosphatidylcholine lipid is distearoylphosphatidylcholine (DSPC) or dioleoylphosphatidylcholine (DOPC).

In another embodiment, the neutral lipid content is between 40 mol % and 60 mol % or between 42 mol % and 60 mol %, or between 45 mol % and 60 mol %, or between 46 mol % and 60 mol %, or between 48 mol % and 60 mol %.

In another embodiment, the ionizable lipid is an amino lipid.

In a further embodiment, the ionizable lipid is present at less than 40 mol %.

In another embodiment, the hydrophilic polymer-lipid conjugate is a polyethyleneglycol-lipid conjugate.

According to a further embodiment, the sterol is present at from 15 mol % to 45 mol % based on the total lipid present in the lipid nanoparticle. In a further embodiment, the sterol is present at from 18 mol % to 40 mol % based on the total lipid present in the lipid nanoparticle.

In another embodiment, the lipid nanoparticle exhibits at least a 10% increase in biodistribution in the spleen, bone marrow, heart, lungs, kidney, abdominal skin, back skin and/or ear as compared to a baseline Onpattro-type formulation of MC3/DSPC/cholesterol/PEG-lipid at 50/10/38.5/1.5, mol:mol encapsulating the oligonucleotide molecule, but otherwise measured under an identical set of conditions, and wherein the biodistribution is quantified in an animal model by detection of labelled lipid at 24 hours post-administration.

In a further embodiment, the oligonucleotide is an antisense oligonucleotide that is single-stranded and has a length of between 30 and 300 nucleotides.

In another embodiment, the oligonucleotide is an siRNA.

In a further aspect, there is provided method for in vivo delivery of an oligonucleotide molecule to a mammalian subject, wherein the oligonucleotide molecule is single-stranded or double-stranded and has a length of between 5 and 500 nucleotides, the method comprising: administering to the mammalian subject the lipid nanoparticle in any one of the foregoing aspects of the disclosure or embodiments thereof.

The present disclosure also provides in one aspect a method for delivering an oligonucleotide molecule to a cell, the method comprising contacting the lipid nanoparticle of any one of the foregoing aspects or embodiments with the cell in vivo or in vitro.

In another embodiment, the oligonucleotide is an antisense oligonucleotide molecule and accumulates in the spleen, bone marrow, heart, lungs and/or kidney of the subject at least one day post-administration.

In yet a further embodiment of the above-described embodiments, the lipid nanoparticle is used to treat a disease or disorder that is an autoimmune disorder.

In another embodiment, the disease or disorder is an infectious disease.

In yet a further embodiment, the disease or disorder is cancer.

In a further aspect of the disclosure, there is provided the lipid nanoparticle of any one of the foregoing aspects or embodiments thereof for in vivo or in vitro delivery of the oligonucleotide molecule to mammalian cells.

According to a further aspect of the disclosure, there is provided a use of the lipid nanoparticle of any one of the aspects or embodiments thereof for the manufacture of a medicament for in vivo or in vitro delivery of the oligonucleotide to mammalian cells.

### BRIEF DESCRIPTION OF THE FIGURES

FIG. 1 is a bar graph showing size (nm), polydispersity index (PDI) and percentage encapsulation of firefly luciferase siRNA of four-component lipid nanoparticles (LNPs) referred to as B1 to B17 comprising the ionizable lipid, MC3, or a sulfur-containing ionizable amino lipid (MF019 as described herein), varying amounts of DSPC ranging from 10 mol % to 55 mol %, cholesterol from 18.9 mol % to 38.5 mol % and PEG₂₀₀₀-DMG at 1.5 mol %. The mol % of each lipid component and nitrogen/phosphate ratio (N/P) in the LNPs labelled B1 to B17 is set forth in Table 1.

FIG. 2A shows images of mice treated with a 40 mol % DSPC-containing LNP (B10 LNP of Table 1) encapsulating firefly luciferase siRNA and having 33/40/25.5/1.5 of MF019 ionizable lipid/DSPC/cholesterol/PEG₂₀₀₀-DMG labelled with DiD lipophilic fluorescent marker and having an N/P of 6. The top panel shows mice before dissection; the middle panels show the abdominal cavity of the mice with organs intact; and the bottom panels show fluorescence in kidney (K), spleen (S), lung (Lu) and liver (Li) excised from the mice.

FIG. 2B shows images of mice treated with a 50 mol % DSPC-containing LNP (B12 LNP of Table 1) encapsulating firefly luciferase siRNA and having 27.4/50/21.1/1.5 of MF019 ionizable lipid/DSPC/cholesterol/PEG₂₀₀₀-DMG labelled with DiD fluorescent marker and having an N/P of 6. The top panel shows mice before dissection; the middle panels show the abdominal cavity of mice with organs intact; and the bottom panels show fluorescence in kidney (K), spleen (S), lung (Lu) and liver (Li) excised from the mice.

FIG. 2C shows images of mice treated with a 40 mol % DSPC-containing LNP (B6 LNP of Table 1) encapsulating firefly luciferase siRNA and having 33/40/25.5/1.5 of MF019 ionizable lipid/DSPC/cholesterol/PEG₂₀₀₀-DMG labelled with DiD marker and having an N/P of 3. The top panel shows mice before dissection; the middle panels show the abdominal cavity of the mice with organs intact; and the bottom panels show fluorescence in kidney (K), spleen (S), lung (Lu) and liver (Li) excised from the mice.

FIG. 3 is a bar graph showing size (nm), polydispersity index (PDI) and percentage encapsulation of firefly luciferase siRNA in four-component lipid nanoparticles (LNPs) referred to as A1 to A16 comprising the ionizable lipid MC3 or a sulfur-containing ionizable amino lipid (MF019 as described herein), varying amounts of egg sphingomyelin (ESM) ranging from 10 mol % to 55 mol %, cholesterol from 18.9 mol % to 38.5 mol % and PEG₂₀₀₀-DMG at 1.5 mol %. The mol % of each lipid component and nitrogen/phosphate ratio (N/P) in LNPs labelled A1 to A16 is set forth in Table 3.

FIG. 4A is a plot showing fluorescent intensity/mg liver homogenate of mice treated with phosphate buffered saline (PBS) control; and firefly luciferase siRNA-containing LNPs having MF019/ESM/cholesterol/PEG₂₀₀₀-DMG at molar ratios 33/40/25.5/1.5 (LNP-A) or 27.4/50/21.1/1.5 (LNP-B) with an N/P of 6; LNPs having MF019/DSPC/cholesterol/PEG₂₀₀₀-DMG at 33/40/25.5/1.5 (LNP-C) or 27.4/50/21.1/1.5 (LNP-D) with an N/P of 6; and 33/40/25.5/1.5 of MF019/DSPC/cholesterol/PEG₂₀₀₀-DMG (LNP-E) with an N/P of 3. The LNPs were labelled with the DiD fluorescent marker.

FIG. 4B is a plot showing fluorescent intensity/mg spleen homogenate of mice treated with a phosphate buffered saline (PBS) control; and firefly luciferase siRNA-containing LNPs having MF019/ESM/cholesterol/PEG₂₀₀₀-DMG at molar ratios 33/40/25.5/1.5 (LNP-A) or 27.4/50/21.1/1.5 (LNP-B) with an N/P of 6; LNPs having MF019/DSPC/cholesterol/PEG₂₀₀₀-DMG at 33/40/25.5/1.5 (LNP-C) or 27.4/50/21.1/1.5 (LNP-D) with an N/P of 6; and 33/40/25.5/1.5 of MF019/DSPC/cholesterol/PEG₂₀₀₀-DMG (LNP-E) with an N/P of 3. The LNPs were labelled with the DiD fluorescent marker.

FIG. 4C is a plot showing fluorescent intensity/mg abdominal skin homogenate of mice treated with a phosphate buffered saline (PBS) control; and firefly luciferase siRNA-containing LNPs having MF019/ESM/cholesterol/PEG₂₀₀₀-DMG at molar ratios 33/40/25.5/1.5 (LNP-A) or 27.4/50/21.1/1.5 (LNP-B) with an N/P of 6; LNPs having MF019/DSPC/cholesterol/PEG₂₀₀₀-DMG at 33/40/25.5/1.5 (LNP-C) or 27.4/50/21.1/1.5 (LNP-D) with an N/P of 6; and 33/40/25.5/1.5 of MF019/DSPC/cholesterol/PEG₂₀₀₀-DMG (LNP-E) with an N/P of 3. The LNPs were labelled with the DiD fluorescent marker.

FIG. 4D is a plot showing fluorescent intensity/mg back skin homogenate of mice treated with a phosphate buffered saline (PBS) control; and firefly luciferase siRNA-containing LNPs having MF019/ESM/cholesterol/PEG₂₀₀₀-DMG at molar ratios 33/40/25.5/1.5 (LNP-A) or 27.4/50/21.1/1.5 (LNP-B) with an N/P of 6; LNPs having MF019/DSPC/cholesterol/PEG₂000-DMG at 33/40/25.5/1.5 (LNP-C) or 27.4/50/21.1/1.5 (LNP-D) with an N/P of 6; and 33/40/25.5/1.5 of MF019/DSPC/cholesterol/PEG₂₀₀₀-DMG (LNP-E) with an N/P of 3. The LNPs were labelled with the DiD fluorescent marker.

FIG. 4E is a plot showing fluorescent intensity/mg ear homogenate of mice treated with a phosphate buffered saline (PBS) control; and firefly luciferase siRNA-containing LNPs having MF019/ESM/cholesterol/PEG₂₀₀₀-DMG at molar ratios 33/40/25.5/1.5 (LNP-A) or 27.4/50/21.1/1.5 (LNP-B) with an N/P of 6; LNPs having MF019/DSPC/cholesterol/PEG₂₀₀₀-DMG at 33/40/25.5/1.5 (LNP-C) or 27.4/50/21.1/1.5 (LNP-D) with an N/P of 6; and 33/40/25.5/1.5 of MF019/DSPC/cholesterol/PEG₂₀₀₀-DMG (LNP-E) with an N/P of 3. The LNPs were labelled with the DiD fluorescent marker.

FIG. 5: In vivo fluorescence of DiD in the liver after treatment with DiD-labelled LNP encapsulating ssRNA as a function of DSPC content. LNPs A and B encapsulate 50 mer ssRNA and LNPs C and D encapsulate 221 mer ssRNA. LNPs A and C are the Onpattro-type baseline composition with 10 mol % DSPC content while B and D are the compositions with 50 mol % DSPC. The Onpattro™ and high DSPC compositions are respectively 50/10/37.75/1.5/0.75 and 27.4/50/20.35/1.5/0.75 mol/mol of ionizable lipid/DSPC/cholesterol/PEG-DMG/DiD.

FIG. 6: In vivo fluorescence of DiD in the spleen after treatment with DiD-labelled LNP encapsulating ssRNA as a function of DSPC content. LNPs A and B encapsulate 50 mer ssRNA and LNPs C and D encapsulate 221 mer ssRNA. LNPs A and C are the Onpattro-type baseline composition with 10 mol % DSPC content while B and D are the compositions with 50 mol % DSPC. The Onpattro-type baseline formulation and high DSPC compositions are respectively 50/10/37.75/1.5/0.75 and 27.4/50/20.35/1.5/0.75 mol/mol of ionizable lipid/DSPC/cholesterol/PEG-DMG/DiD.

FIG. 7: In vivo fluorescence of DiD in the bone marrow (BM) after treatment with DiD-labelled LNP encapsulating ssRNA as a function of DSPC content. LNPs A and B encapsulate 50 mer ssRNA and LNPs C and D encapsulate 221 mer ssRNA. LNPs A and C are the Onpattro-type composition with 10 mol % DSPC content while B and D are the compositions with 50 mol % DSPC. The Onpattro-type baseline and high DSPC compositions are respectively 50/10/37.75/1.5/0.75 and 27.4/50/20.35/1.5/0.75 mol/mol of ionizable lipid/DSPC/cholesterol/PEG-DMG/DiD.

FIG. 8: In vivo fluorescence of DiD in the heart after treatment with DiD-labelled LNP encapsulating ssRNA as a function of DSPC content. LNPs A and B encapsulate 50 mer ssRNA and LNPs C and D encapsulate 221 mer ssRNA. LNPs A and C are the Onpattro-type baseline composition with 10 mol % DSPC content while B and D are the compositions with 50 mol % DSPC. The Onpattro-type baseline and high DSPC compositions are respectively 50/10/37.75/1.5/0.75 and 27.4/50/20.35/1.5/0.75 mol/mol of ionizable lipid/DSPC/cholesterol/PEG-DMG/DiD.

FIG. 9: In vivo fluorescence of DiD in the lung after treatment with DiD-labelled LNP encapsulating ssRNA as a function of DSPC content. LNPs A and B encapsulate 50 mer ssRNA and LNPs C and D encapsulate 221 mer ssRNA. LNPs A and C are the Onpattro-type baseline composition with 10 mol % DSPC content while B and D are the compositions with 50 mol % DSPC. The Onpattro-type baseline and high DSPC compositions are respectively 50/10/37.75/1.5/0.75 and 27.4/50/20.35/1.5/0.75 mol/mol of ionizable lipid/DSPC/cholesterol/PEG-DMG/DiD.

FIG. 10: In vivo fluorescence of DiD in the kidney after treatment with DiD-labelled LNP encapsulating ssRNA as a function of DSPC content. LNPs A and B encapsulate 50 mer ssRNA and LNPs C and D encapsulate 221 mer ssRNA. LNPs A and C are the Onpattro-type baseline composition with 10 mol % DSPC content while B and D are the compositions with 50 mol % DSPC. The Onpattro-type baseline and high DSPC compositions are respectively 50/10/37.75/1.5/0.75 and 27.4/50/20.35/1.5/0.75 mol/mol of ionizable lipid/DSPC/cholesterol/PEG-DMG/DiD.

FIG. 11A: Cryo-TEM images of siRNA-containing LNPs encapsulating firefly luciferase siRNA and having 33/40/25.5/1.5 of MF019 ionizable lipid/DSPC/cholesterol/PEG₂₀₀₀-DMG with an N/P of 6.

FIG. 11B: Cryo-TEM images of siRNA-containing LNPs encapsulating firefly luciferase siRNA and having 27.4/50/21.1/1.5 of MF019 ionizable lipid/DSPC/cholesterol/PEG₂₀₀₀-DMG with an N/P of 6.

FIG. 11C: Cryo-TEM images of siRNA-containing LNPs encapsulating firefly luciferase siRNA and having 27.4/50/21.1/1.5 of MF019 ionizable lipid/ESM/cholesterol/PEG₂₀₀₀-DMG with an N/P of 6.

### DETAILED DESCRIPTION

The lipid nanoparticles described herein provide improvements in the delivery of oligonucleotide cargo over the conventional four-component LNP, referred to herein as an Onpattro-type LNP. In particular, the LNP comprises ionizable lipid, neutral lipid, such as phosphatidylcholine lipid (e.g., DSPC), sterol and optionally a hydrophilic polymer-lipid conjugate, and in which the neutral lipid (e.g., phosphatidylcholine lipid) is present at a mol % of at least 20 mol %, at least 25 mol % or at least 30 mol % and in which the ionizable lipid, in some examples, is present at less than 40 mol %. As set forth herein, the inclusion of neutral lipid, such as phosphatidylcholine at a mol % higher than that used in conventional formulations for nucleic acid delivery provides selective delivery to extrahepatic tissues relative to Onpattro-type baseline.

The “oligonucleotide” or “oligonucleotide cargo” is a single-stranded or double-stranded RNA or DNA molecule and has a length of between 5 and 500 nucleotides. The term includes an antisense oligonucleotide (ASO) that is single stranded and generally 30 to 500 nucleotides in length or a shorter length, double stranded silencing RNA molecule (siRNA), which is 3 to 40 nucleotides in length.

### Neutral Lipid

The neutral lipid is an amphipathic lipid that allows for the formation of particles and generally bears no net charge at physiological pH. The term includes zwitterionic lipids and examples include phospholipids.

In some embodiments, the neutral lipid is a phosphatidycholine lipid. The phosphatidylcholine lipid may be selected from distearoylphosphatidylcholine (DSPC), dioleoylphosphatidylcholine (DOPC), 1-palmitoyl-2-oleoyl-phosphatidylcholine (POPC), dimyristoylphosphatidylcholine (DMPC) and dipalmitoyl-phosphatidylcholine (DPPC). The phosphatidylcholine lipid component may include mixtures of two or more types of different neutral lipids.

In one embodiment, the phosphatidylcholine lipid is selected from DSPC, POPC and mixtures thereof.

The phosphatidylcholine content in some embodiments is greater than 20 mol %, greater than 25 mol %, greater than 30 mol %, greater than 32 mol %, greater than 34 mol %, greater than 36 mol %, greater than 38 mol %, greater than 40 mol %, greater than 42 mol %, greater than 44 mol %, greater than 46 mol %, greater than 48 mol % or greater than 50 mol %. In some embodiments, the upper limit of neutral lipid content is 70 mol %, 65 mol %, 60 mol %, 55 mol %, 50 mol % or 45 mol %. The disclosure also encompasses sub-ranges of any combination of the foregoing numerical upper and lower limits.

For example, in certain embodiments, the phosphatidylcholine lipid content is from 20 mol % to 80 mol % or 25 mol % to 60 mol % or 30 mol % to 60 mol % or 35 mol % to 60 mol % or 40 mol % to 60 mol % or 42 mol % to 58 mol %, or 43 mol % to 57 mol % or 44 mol % to 56 mol % or 45 mol % to 55 mol % of total lipid present in the lipid nanoparticle.

In one embodiment, most advantageously, the neutral lipid is DSPC. In some examples of the disclosure, the DSPC lipid at elevated improves the biodistribution of the LNP over other neutral phospholipids. In certain embodiments, the DSPC lipid content is from 20 mol % to 80 mol % or 25 mol % to 60 mol % or 30 mol % to 60 mol % or 35 mol % to 60 mol % or 40 mol % to 60 mol % or 42 mol % to 60 mol %, or 43 mol % to 60 mol % or 44 mol % to 60 mol % or 45 mol % to 60 mol % or 46 mol % to 60 mol % or 48 mol % to 60 mol % of total lipid present in the lipid nanoparticle.

In some embodiments, the phosphatidylcholine lipid is a sphingolipid. As used herein the term “sphingolipid”, means a lipid comprising a sphingosine backbone and that is suitable for formulation in the LNPs herein. The sphingolipid includes a ceramide, a sphingomyelin, a cerebroside, a ganglioside, or derivatives, such as but not limited to reduced analogues thereof, that lack a double bond in the sphingosine unit. The sphingolipid has a phosphocholine head group and includes sphingomyelin.

In certain embodiments, the sphingolipid lipid content is from 20 mol % to 80 mol % or 25 mol % to 60 mol % or 30 mol % to 60 mol % or 35 mol % to 60 mol % or 40 mol % to 60 mol % or 42 mol % to 60 mol %, or 43 mol % to 60 mol % or 44 mol % to 60 mol % or 45 mol % to 60 mol % or 46 mol % to 60 mol % or 48 mol % to 60 mol % of total lipid present in the lipid nanoparticle.

The sphingomyelin content of the lipid nanoparticle in some examples is less than 5 mol %, less than 4 mol %, less than 3 mol %, less than 2 mol %, less than 1 mol %, less than 0.75 mol %, or less than 0.5 mol %. In some embodiments, the LNP is “sphingomyelin-free”, meaning there is no detectable sphingomyelin in the LNP (less than 0.5 mol %) or the LNP is substantially sphingomyelin-free, meaning there is less than 5 mol % or 2.5 mol % sphingomyelin in the LNP.

The LNP may comprise additional neutral lipids besides a phosphatidylcholine lipid. For example, the LNP may comprise other lipids that have a net positive or negative charge at physiological pH. In another example, the LNP may further comprise lesser amounts of one or more fusogenic lipids (relative to the phosphatidylcholine lipids), such as DOPE, which are cone-shaped and thereby promote fusion with a cell membrane. Generally, such lipids will be present at less than 10 mol %, less than 9 mol %, less than 8 mol %, less than 7 mol %, less than 6 mol % or less than 5 mol % relative to total lipid present in the LNP.

The inclusion of fusogenic lipids, such as dioleoylphosphatidylethanolamine (DOPE) is thought to facilitate nucleic acid delivery in vitro or in vivo. However, the present disclosure, in some examples, generally does not favour the inclusion of such lipids. Accordingly, the fusogenic lipid content of the lipid nanoparticle in some examples is less than 5 mol %, less than 4 mol %, less than 3 mol %, less than 2 mol %, less than 1 mol %, less than 0.75 mol %, or less than 0.5 mol %. In some embodiments, the LNP is “fusogenic lipid-free”, meaning there are no detectable amounts of fusogenic lipids present in the LNP (less than 0.5 mol %) or the LNP is substantially fusogenic lipid-free, meaning there is less than 5 mol % fusogenic lipid content measured relative to total lipid content in the LNP.

The DOPE content of the lipid nanoparticle in some examples is less than 10 mol %, less than 8 mol %, 5 mol %, less than 4 mol %, less than 3 mol %, less than 2 mol %, less than 1 mol %, less than 0.75 mol %, or less than 0.5 mol %. In some embodiments, the LNP is “DOPE-free”, meaning there is no detectable DOPE in the LNP (less than 0.5 mol %) or the LNP is substantially DOPE-free, meaning there is less than 5 mol % DOPE measured relative to total lipid content in the LNP.

In further embodiments, it might be advantageous to include mixtures of different phosphatidylcholine lipids in the LNP. However, in some examples, the phosphatidylcholine lipid content includes less than 5, 4, or 3 different phosphatidylcholine lipids.

The egg phosphatidylcholine (EPC) content of the lipid nanoparticle in some examples is less than 5 mol %, less than 4 mol %, less than 3 mol %, less than 2 mol %, less than 1 mol %, less than 0.75 mol %, or less than 0.5 mol %. In some embodiments, the LNP is “EPC-free”, meaning there is no detectable EPC (less than 0.5 mol %) in the LNP or the LNP is substantially EPC-free, meaning there is less than 5 mol % EPC in the lipid nanoparticle measured relative to total lipid content in the LNP.

In some embodiments, the phosphatidylcholine lipid is a phosphatidylcholine-sterol conjugate, such as an SPC-cholesterol, OPC-cholesterol or PPC-cholesterol conjugate. Additional phospholipid-sterol conjugates are described in US2011/0177156, which is incorporated herein by reference. An example of a suitable phospholipid that is an SPC-cholesterol conjugate is set forth below:

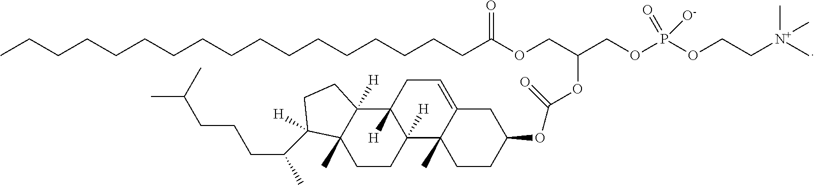

In some embodiments, the transition temperature of the neutral lipid, or mixture thereof, is at least 20° C., 21° C., 22° C., 23° C., 24° C., 25° C., 26° C., 27° C., 28° C., 29° C., 30° C., 31° C., 32° C., 33° C., 34° C., 35° C., 36° C., 37° C. or 38° C. In one embodiment, the phase transition temperature of the neutral lipid, or mixture thereof, when incorporated in the lipid nanoparticle is at least 38, 39 or 40 degrees Celsius.

The neutral lipid content is determined based on the total amount of lipid in the lipid nanoparticle, including the sterol (mol:mol).

### Ionizable Lipid

The LNP of the disclosure has an ionizable cationic lipid, which includes one or a combination of two or more of such lipids.

As used herein, the term “ionizable cationic lipid” refers to a lipid that, at a given pH, such as physiological pH, is in an electrostatically neutral form and that accepts protons, thereby becoming electrostatically positively charged at a pH below its pKa. The electrostatically neutral form has a calculated logarithm of the partition coefficient between water and 1-octanol (i.e., a c Log P) greater than 8. In some embodiments, the cationic lipid has a pKa that is between 5.0 and 7.5 or between 6.0 and 7.5 when formulated in the LNP.

Accordingly, the ionizable lipid may be charged at low pH and have substantially no net charge at physiological pH. This allows for electrostatic interactions between the lipid and the negatively charged nucleic acid cargo during initial formulation. Since the ionizable lipid is near neutral at physiological pH, toxicity and renal clearance is reduced. Without being limited by theory, after cellular uptake by endocytosis, the acidic environment of the endosome leads to an increase in the net positive charge of the ionizable amino lipids, which promotes fusion with the anionic lipids of the endosomal membrane and subsequent membrane destabilization and release of the nucleic acid-based therapeutics into the cytoplasm to exert their effects.

In some embodiments, the LNP has an apparent pKa of between 5.0 and 7.5, between 6.5 and 7.5 or between 6.8 and 7.3. The apparent pKa is measured using a 6-(p-Toluidino)-2-naphthalenesulfonic acid (TNS) assay adapted from previous studies from other groups (Shobaki et al., 2018, International Journal of Nanomedicine, 13:8395-8410; and Jayaraman et al., 2012, Angew. Chem Int. Ed., 51:8529-8533, which are incorporated herein by reference for the purposes of determining apparent pKa). According to the method, a series of buffers are prepared spanning a pH range of 2-11 in 0.5 pH unit increments consisting of 130 mM NaCl, 10 mM ammonium acetate, 10 mM 2-(N-morpholino)ethanesulfonic acid (MES), and 10 mM HEPES. 0.15-0.2 mM of the LNP. A solution of 0.06 mM of TNS is subsequently mixed with 175 μL of the LNP at each buffered pH in triplicate in a black, polysterene 96-well plate, to yield a final concentration of 6.25 and 6 μM of lipid and TNS in each well, respectively. Fluorescence is subsequently measured using an SpectraMax™ M5 microplate reader at λₑₓ=321 nm, λₑₘ=445 nm. The fluorescence is then plotted against pH using a sigmoidal curve fit through Prism™, in which the pKa is determined to be the pH value with 50% of maximal fluorescent intensity.

In some embodiments, it is desirable to include less than 50 mol % ionizable lipid in the LNP. That is, the ionizable lipid content may be less than 50 mol %, less than 45 mol %, less than 40 mol %, less than 35 mol %, less than 30 mol %, less than 25 mol %, less than 20 mol %, less than 15 mol %, less than 10 mol % or less than 5 mol % as measured based on total lipid content of the LNP. In certain embodiments, the lower limit of the ionizable lipid content may be greater than 5 mol %, greater than 8 mol %, greater than 10 mol %, greater than 12 mol %, greater than 14 mol %, greater than 15 mol %, greater than 16 mol %, greater than 18 mol %, greater than 20 mol %, greater than 25 mol % or greater than 30 mol %. Any one of the upper limits may be combined with any one of the lower limits to arrive at a suitable ionizable lipid content in the LNP.

In certain embodiments, the ionizable lipid content is from 5 mol % to 50 mol % or 8 mol % to 47 mol % or 10 mol % to 50 mol % or 15 mol % to 45 mol % or 15 mol % to 35 mol % of total lipid present in the lipid nanoparticle.

In some embodiments, the cationic lipid has an amino group. In some cases, the cationic lipid comprises a protonatable tertiary amine (e.g., pH titratable) head group and two C16 to C18 alkyl chains having 0 to 3 double bonds. Such lipids include, but are not limited to sulfur lipids, such as MF019 described herein and DODMA. Other lipids that may be used in the practice of the disclosure include MC3- and KC2-type lipids, which are well-known to those of skill in the art. In further embodiments, the ionizable lipid is selected from one or more lipids set forth in WO 2022/246555; WO 2022/246568; WO 2022/246571; WO 2023/147657; WO2022/155728; PCT/CA2023/050644 filed on May 11, 2023; PCT/CA2023/051272 filed on Sep. 27, 2023; PCT/CA2023/051273 filed on Sep. 27, 2023; U.S. provisional patent application No. 63/434,506 filed on Dec. 22, 2022; PCT/CA2023/051274 filed on Sep. 27, 2023; and U.S. provisional patent application No. 63/445,854 filed on Feb. 15, 2023, each incorporated herein by reference.

In some embodiments, it is desirable to include less than 50 mol % ionizable cationic lipid in the LNP. That is, the ionizable cationic lipid content may be less than 50 mol %, less than 45 mol %, less than 40 mol %, less than 35 mol %, less than 30 mol %, less than 25 mol %, less than 20 mol %, less than 15 mol %, less than 10 mol % or less than 5 mol %.

In certain embodiments, the ionizable cationic lipid content is from 5 mol % to 50 mol % or 8 mol % to 47 mol % or 10 mol % to 50 mol % or 15 mol % to 45 mol % or 15 mol % to 35 mol % of total lipid present in the lipid nanoparticle.

In certain embodiments, the ionizable amino cationic lipid content is from 15 mol % to 40 mol % or 8 mol % to 47 mol % or 10 mol % to 50 mol % or 15 mol % to 45 mol % or 15 mol % to 35 mol % of total lipid present in the lipid nanoparticle.

In some embodiments, the ionizable cationic lipid is not a lipidoid structure, including but not limited to C12-200 (see Khare et al., 2022, AAPS Journal, 24:8, incorporated by reference) and related structures known to those of skill in the art.

### Sterol

The LNP further includes a sterol in some embodiments. The term “sterol” refers to a naturally-occurring or synthetic compound having a gonane skeleton and that has a hydroxyl moiety attached to one of its rings, typically the A-ring.

Examples of sterols include cholesterol, or a cholesterol derivative, the latter referring to a cholesterol molecule having a gonane structure and one or more additional functional groups.

The cholesterol derivative includes β-sitosterol, 3-sitosterol, campesterol, stigmasterol, fucosterol, or stigmastanol, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β[N—(N′N′-dimethylaminoethyl)carbamoyl cholesterol (DC-Chol), 24(S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol or a salt or ester thereof.

In one embodiment, the sterol is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is cholesterol and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is a cholesterol derivative and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In one embodiment, the combined (i) sterol content (e.g., cholesterol or cholesterol derivative thereof); and (ii) phosphatidylcholine lipid content is at least 50 mol %; at least 55 mol %, at least 60 mol %, at least 65 mol %, at least 70 mol %, at least 75 mol %, at least 80 mol % or at least 85 mol % based on the total lipid present in the lipid nanoparticle.

### Hydrophilic Polymer-Lipid Conjugate

In one embodiment, the lipid nanoparticle comprises a hydrophilic-polymer lipid conjugate capable of incorporation into the LNP. In some examples, the conjugate includes a vesicle-forming lipid having a polar head group, and covalently attached to the head group, a polymer chain that is hydrophilic. The lipid includes any moiety that has at least a hydrophobic portion. Examples of hydrophilic polymers include polyethyleneglycol (PEG), polyvinylpyrrolidone, polyvinylmethylether, polyhydroxypropyl methacrylate, polyhydroxypropylmethacrylamide, polyhydroxyethyl acrylate, polymethacrylamide, polydimethylacrylamide, polymethyloxazoline, polyethyloxazoline, polyhydroxyethyloxazoline, polyhydroxypropyloxazoline, polysarcosine and polyaspartamide. In one embodiment, the hydrophilic-polymer lipid conjugate is a PEG-lipid conjugate. The hydrophilic polymer lipid conjugate may also be a naturally-occurring or synthesized oligosaccharide-containing molecule, such as monosialoganglioside (G_{M1}). The ability of a given hydrophilic-polymer lipid conjugate to enhance the circulation longevity of the LNPs herein could be readily determined by those of skill in the art using known methodologies.

The hydrophilic polymer lipid conjugate may be present in the nanoparticle at 0.5 mol % to 5 mol %, or at 0.5 mol % to 3 mol %, or at 0.5 mol % to 2.5 mol % or at 0.5 mol % to 2.0 mol % or at 0.5 mol % to 1.8 mol % of total lipid. In certain embodiments, the hydrophilic polymer lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In another embodiment, the PEG-lipid conjugate is present in the nanoparticle at 0.5 mol % to 5 mol %, or at 0.5 mol % to 3 mol % or at 0.5 mol % to 2.5 mol % or at 0.5 mol % to 2.0 mol % or at 0.5 mol % to 1.8 mol % of total lipid. In certain embodiments, the PEG-lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In one embodiment, the hydrophilic polymer-lipid is selected based on its exchangeability from the lipid nanoparticles. Such property may facilitate in vivo efficacy due to at least partial loss of the hydrophilic polymer-lipid conjugate from the LNP as it reaches a target site in vivo.

In such embodiment, the lipid moiety of the hydrophilic polymer-lipid conjugate typically has acyl chain lengths of less than 18 and having 0-2 double bonds in one or both of the acyl chains. In some embodiments, the hydrophilic polymer-lipid conjugate is a PEG-lipid conjugate selected from dimyristoylphosphatidylethanolamine-PEG (DMPE-PEG), dipalmitoylphosphatidylethanolamine-PEG (DPPE-PEG), dioleylphosphatidylethanolamine-PEG (DOPE-PEG), dipalmitoylphosphatidylethanolamine-PEG (DPPE-PEG), dimyristoyldiglyceride-PEG (DMG-PEG) or cholesterol-PEG (Chol-PEG).

In one embodiment, the hydrophilic polymer lipid conjugate is not present or present at low concentration in the lipid nanoparticle. In some embodiments, the hydrophilic polymer-lipid conjugate content is less than 0.5 mol %, 0.45 mol %, 0.40 mol %, 0.35 mol %, 0.30 mol %, 0.25 mol %, 0.20 mol % or 0.15 mol %.

In one embodiment, the hydrophilic polymer lipid conjugate is not DSPE-PEG. In further embodiments, the DSPE-PEG content is less than 0.5 mol %, 0.45 mol %, 0.40 mol %, 0.35 mol %, 0.30 mol %, 0.25 mol %, 0.20 mol % or 0.15 mol %.

In a further embodiment, within the hydrophilic polymer lipid conjugate a cleavable linker is present between the lipid moiety and the hydrophilic polymer. Such linkers may be cleavable by exposure to low pH, reducing agents or proteases present in vivo. Examples of cleavable linkers include esters, ethers, phosphoroamidate, hydrazone, beta-thiopropionate, disulfide groups and peptides (Romberg et al., 2008, Pharmaceutical Research, 25:55-71, incorporated herein by reference).

### Optional Additional Lipid Components

In some embodiments, the lipid nanoparticle for extrahepatic delivery includes lipids “consisting essentially of” a neutral amphipathic lipid such as a phosphocholine, a sterol or derivative thereof, an ionizable cationic lipid and optionally a hydrophilic polymer-lipid conjugate and has less than 10 mol % of additional lipid components beyond the foregoing lipid components. That is, in some examples, the additional lipid components are present at 0-20 mol %, 0-15 mol %, 0-10 mol %, 0 to 8 mol % or 0 to 5 mol % and include any one of a number of charged and/or uncharged lipids. In some embodiments, the additional lipid component consists of one or more neutral lipids rather than lipids that are charged at physiological pH. Avoiding such charged lipids may reduce uptake by the RES system and thereby not compromise the circulation lifetime of the particle. Examples of such optional additional lipids include triacylglycerides, diacylglycerides, monoacylglycerides, zwitterionic lipids, antioxidants and vitamins.

Examples of additional phospholipids include phosphatidyl glycerols, such as dioleoylphosphatidylglycerol (DOPG), distearoylphosphatidylethanolamine (DSPE), dioleoylphosphatidylethanolamine (DOPE), distearoylphosphatidylserine (DSPS) and sphingomyelins. Examples of sphingomyelins in this context include a ceramide, a sphingomyelin, a cerebroside, a ganglioside, or reduced analogues thereof, that lack a double bond in the sphingosine unit.

In those embodiments in which an anionic lipid is included in the formulation, the anionic lipid may be a lipid with a head group that has a hydroxyl group. Without limitation, this includes anionic lipids such as DOPG and A-0001, which is described in U.S. provisional patent application No. 63/453,766 filed on Mar. 22, 2023 (incorporated herein by reference).

Further additional components include hydrophobic moieties such as lipids conjugated to targeting ligands. The ligand includes peptides, polypeptides or proteins and includes antibodies or fragments thereof. In one embodiment, the ligand may be a single-chain antibody fragment. The targeting ligand may be used to target receptors on cells in vivo. In some embodiments, the targeting ligand may be conjugated to a phospholipid component of the LNP. If some amounts of hydrophilic polymer lipid conjugate is present, a targeting ligand may be conjugated to its distal end. In such embodiments, the phospholipid-targeting ligand conjugate is present at less than 10 mol %, less than 5 mol % or less than 3 mol %. In some embodiments, the targeting ligand is most advantageously absent.

In one embodiment, the lipid nanoparticle has low levels (less than 5 mol %) or no permanently charged cationic lipid content. In some examples of the disclosure, the lipid nanoparticles, due to the lack of a net charge at physiological pH, promote extended circulation longevity, thereby facilitating extrahepatic delivery to cells. Examples of permanently charged cationic lipids that are best avoided include dioctadecyldimethylammonium bromide (DDAB), and 1,2-Dioleoyl-3-trimethylammonium propane (DOTAP). It is also possible that such permanently charged cationic lipids could impart toxicity to the lipid nanoparticles.

### Nanoparticle Preparation and Morphology

Lipid nanoparticles incorporating the oligonucleotide can be prepared using a variety of suitable methods, such as a rapid mixing/ethanol dilution process. Examples of preparation methods are described in Jeffs, L. B., et al., Pharm Res, 2005, 22(3):362-72; and Leung, A. K., et al., The Journal of Physical Chemistry. C, Nanomaterials and Interfaces, 2012, 116(34): 18440-18450, each of which is incorporated herein by reference in its entirety.

Without being bound by theory, the mechanism whereby a lipid nanoparticle comprising encapsulated oligonucleotide can be formed using the rapid mixing/ethanol dilution process can be hypothesized as beginning with formation of a dense region of hydrophobic RNA-ionizable lipid core at low pH (e.g., pH 4) surrounded by a monolayer of neutral lipid/cholesterol that fuses with smaller empty vesicles as the pH is raised due to the conversion of the ionizable cationic lipid to the neutral form. As the proportion of bilayer neutral lipid increases, the bilayer lipid progressively forms blebs and the ionizable lipid migrates to the interior hydrophobic core. At high enough neutral lipid contents, the exterior bilayer preferring neutral lipid can form a complete bilayer around the interior trapped volume.

The LNP may comprise a “core” region. The core may be considered non-homogeneous in that it includes an electron dense region and optionally an aqueous portion at least partially surrounding the electron dense region. The electron dense region is visualized by cryo-EM microscopy using the procedure described in the Materials and Methods herein. Without being limiting, the electron dense region within the core may be partially surrounded by the aqueous portion within the enclosed space as observed by cryo-TEM. The aqueous portion forms a distinct aqueous compartment within the lipid nanoparticle. In other words, the aqueous portion is not merely a hydration layer.

In one embodiment, at least one about fifth of the core (trapped volume) contains the aqueous portion, and in which the electron dense region is partially contiguous with the lipid layer comprising the bilayer, as determined qualitatively by cryo-EM. In another embodiment, at least one about quarter of the core contains the aqueous portion, and in which the electron dense core is partially contiguous with the lipid layer comprising the bilayer, as determined qualitatively by cryo-EM. In a further embodiment, at least one about one third of the core contains the aqueous portion, and in which the electron dense region is partially contiguous with the lipid layer comprising the bilayer, as determined qualitatively by cryo-EM. In another embodiment, at least one about one half of the core contains the aqueous portion, and in which the electron dense core is partially contiguous with the lipid layer comprising the bilayer, as determined qualitatively by cryo-EM.

In one embodiment, the electron dense region is generally spherical in shape. In another embodiment, the electron dense region is hydrophobic.

The lipid nanoparticles herein may exhibit particularly high encapsulation efficiencies of oligonucleotide. As used herein, the term “encapsulation,” with reference to incorporating the oligonucleotide within a lipid nanoparticle refers to any association of the oligonucleotide with any lipid component or compartment of the lipid nanoparticle, including a lipophilic or the aqueous portion. In one embodiment, the oligonucleotide is present at least in the core of the LNP.

In one embodiment, the encapsulation efficiency is at least 50, 55, 60, 65, 70, 75, 80, 85, 90% or 92%. The encapsulation efficiency of the oligonucleotide is determined as set forth in the Materials and Methods section in the Examples herein.

The lipid nanoparticle may comprise a single bilayer or comprise multiple lipid layers (i.e., multi-lamellar). The one or more lipid layers, including the bilayer, may form a continuous layer surrounding the core or may be discontinuous. The lipid layer may be a combination of a bilayer and a monolayer in some embodiments. In one embodiment, the lipid layer is a continuous bilayer that surrounds the core.

Thus, in certain embodiments the electron dense region of the core is separated from the lipid layer comprising the bilayer by the aqueous portion. For example, the disclosure provides a lipid nanoparticle preparation comprising a plurality of lipid nanoparticles in which at least 10%, 20%, 30%, 40%, 50%, 60% or 70% of the particles as determined by cryo-EM microscopy have a core with an electron dense region that is partially surrounded by the aqueous portion and in which the aqueous portion is partially surrounded by the lipid layer comprising the bilayer as visualized by cryo-EM microscopy.

In another embodiment, and without being limiting, the disclosure provides a lipid nanoparticle preparation comprising a plurality of lipid nanoparticles in which generally at least 10%, 20%, 30%, 40%, 50%, 60% or 70% of the particles have an elongate shape (e.g., generally oval-shaped) as determined qualitatively by cryo-EM microscopy. In this latter embodiment, the electron dense region of the core may be partially surrounded by the aqueous space as visualized by cryo-EM microscopy.

In one embodiment, the lipid nanoparticle is part of a preparation of lipid nanoparticles, and wherein the electron dense region of at least 20% of the lipid nanoparticles are either (i) enveloped by the aqueous portion, or (ii) is partially surrounded by the aqueous portion and wherein a portion of a periphery of the electron dense region is contiguous with the lipid layer, as visualized by cryo-EM microscopy.

In certain embodiments, the disclosure provides a lipid nanoparticle preparation comprising a plurality of lipid nanoparticles in which generally at least 10%, 20%, 30%, 40%, 50%, 60% or 70% of the particles as determined by cryo-EM microscopy have a core with an electron dense region that is contiguous with the lipid layer comprising the bilayer as visualized by cryo-EM microscopy.

In another embodiment, and without being limiting, the disclosure provides a lipid nanoparticle preparation comprising a plurality of lipid nanoparticles in which generally at least 10%, 20%, 30%, 40%, 50%, 60% or 70% of the particles have a core comprising an electron dense region surrounded or enveloped by a continuous aqueous space disposed between the lipid layer (e.g., bilayer) and the electron dense region as visualized by cryo-EM microscopy.

LNPs may be visualized by cryo-TEM as described in the Example section hereinafter.

In another embodiment, the polydispersity index (PDI) of the LNP preparation is less than 0.3, 0.25, 0.2, 0.15, 0.12 or 0.10.

In another embodiment, the particle size distribution is such that 90% of the particles in the LNP preparation of the disclosure have a diameter of between 40 nm and 200 nm, between 40 nm and 150 nm, between 40 nm and 140 nm, between 45 and 150 nm, between 50 nm and 120 nm or between 50 and 140 nm. In some embodiments, the LNPs herein have a PDI of less than 0.25, less than 0.20, less than 0.18, less than 0.16, less than 0.15 or less than 0.14.

Embodiments of the present disclosure also provide lipid nanoparticles described according to the molar ratio between the positively charged amine groups of the amine lipid (N) and the negatively charged phosphate groups (P) of the oligonucleotide to be encapsulated. This may be mathematically represented by the equation N/P. In one embodiment, the N/P ratio of the lipid nanoparticle is between 4 and 15 or between 4.5 and 10 or between 5 and 10 or between 5.5 and 8.

In one embodiment, the N/P ratio of the lipid nanoparticle is at least 4, 4.25, 4.50, 4.75, 5.0, 5.25, 5.5, 5.75, 6.0 or 6.25. The upper limit may be 15, 14, 13, 12, 11, 10, 9 or 8. The disclosure also encompasses a combination of any two of the upper and lower limits. The N/P is a charge ratio that is calculated as set forth in the Materials and Methods.

In one embodiment, the lipid nanoparticle has a weight nucleic acid/micromole of total lipid that is 0.05:1 to 1:1. In one embodiment, the lower limit is 0.06:1, 0.08:1, 0.10:1, 0.12:1, 0.14:1, 0.16:1, 0.18:1, 0.20:1, 0.22:1, 0.24:1, 0.26:1, 0.28:1, 0.30:1, 0.32:1, 0.34:1, 0.36:1, 0.38:1 or 0.40:1 weight nucleic acid/micromole of total lipid. In another embodiment, the upper limit is 0.80:1, 0.82:1, 0.84:1, 0.86:1, 0.88:1, 0.90:1, 0.92:1, 0.94:1, 0.96:1 or 0.98:1 weight nucleic acid/micromole of total lipid. The disclosure also encompasses a combination of any two of the upper and lower limits.

### Oligonucleotide Cargo

The oligonucleotide cargo includes interfering RNA and antisense oligonucleotides described in more detail hereinafter.

### Interfering RNA

The oligonucleotide in one embodiment is a “short interfering RNA” or “siRNA”, which is an RNA molecule capable of reducing or inhibiting the expression of a target gene or nucleic acid sequence in a cell. In one embodiment, the short interfering RNA may mediate the degradation of a target mRNA as measured in vitro or in vivo. In such embodiment, the siRNA may function via base-pairing (when single-stranded) with complementary sequences of a target mRNA and induce mRNA cleavage.

The siRNA is double stranded and may be of a variety of lengths but is generally less than 35 nucleotides in length. In some embodiments, the siRNA has a length such as 1 to 35 nucleotides in length or 15 to 30 nucleotides in length or 20 to 25 nucleotides in length.

In those embodiments in which the siRNA reduces expression of a target gene or sequence by complementary base pairing and degradation of mRNA, the siRNA may have substantial or complete identity to the gene that encodes a target sequence, or may comprise a region of mismatch (i.e., a mismatch motif). The sequence of the siRNA can correspond to the full-length target sequence, or a subsequence thereof.

The double-stranded siRNA encapsulated in the LNP may include duplex RNA, such as double stranded small interfering RNA, asymmetrical interfering RNA (aiRNA) or pre-miRNA or a hybrid molecule comprising both RNA and DNA. In one embodiment, the double-stranded RNA is self-complementary. In such embodiments, the siRNA may form a stem loop or hairpin structure at one end.

The siRNA encompassed by embodiments of the disclosure may be used to inhibit expression of a wide range of target polynucleotides. The siRNA molecule targeting a specific polynucleotide for any therapeutic, prophylactic or diagnostic application may be readily prepared according to procedures known in the art. An siRNA target site may be selected and corresponding interfering RNAs may be chemically synthesized, created by in vitro transcription, or expressed from a vector or PCR product.

As noted, the siRNA described herein may comprise a “mismatch motif” or “mismatch region”, which refers to a portion of the siRNA sequence that does not have 100% complementarity to its target sequence. An siRNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

The nucleotides of the siRNA may or may not be chemically modified. Examples of optional modifications include, but are not limited to, 2′-O-alkyl modifications such as 2′-O-Me or 2′-O-methoxyethyl modifications and 2′-halogen modifications such as 2′-fluoro modifications. In yet further embodiments, the siRNA comprises one, two, three, four, or more 2′-deoxy nucleotides, e.g., in the sense and/or antisense strand of the double-stranded region. Alternately or additionally, in some embodiments, the siRNA comprises phosphate backbone modifications.

Within an siRNA, the antisense strand and the sense strand may be designed such that when they form a duplex due to complementarity base pairing, they can anneal with no overhangs and thus form blunt ends at both ends of the duplex, or with an overhang at one or more of the 3′ end of the sense strand, the 3′ end the antisense strand, the 5′ end of the sense strand, and the 5′ end of the antisense strand. In some embodiments, there are no 5′ overhangs and there is no 3′ antisense overhang, but there is a 3′ sense overhang. In other aspects, there are no 5′ overhangs, but there is a 3′ antisense overhang and a 3′ sense overhang. The overhangs may comprise T or U nucleotides.

In some embodiments, the siRNA is covalently bound to one or more other moieties to form a conjugate. In some embodiments, the conjugates are selected based on their ability to facilitate delivery of the siRNA to an organism or into cells. An siRNA may be bound to a moiety at, for example, the 5′ end of the antisense strand, the 3′ end of the antisense strand, the 5′ end of the sense strand, the 3′ end of the sense strand, or to a nucleotide at a position that is not at the 3′ end or 5′ end of either strand.

Examples of conjugates include but are not limited to one or more of an antibody or fragments thereof, a peptide, an amino acid, an aptamer, a phosphate group, a cholesterol moiety, a lipid, a cell-penetrating peptide, a polymer, and a sugar group, which includes a sugar monomer, an oligosaccharide and modifications thereof. In one non-limiting example, the conjugate is N-Acetylgalactosamine (GalNAc).

### Anti-Sense Oligonucleotide (ASO)

The nucleic acid cargo in one embodiment is an “antisense oligonucleotide” or “ASO”, which is a single strand of nucleic acid (e.g., RNA or DNA) that binds to a target nucleic acid sequence by base pairing. The ASO may have substantial or complete identity to the gene that encodes a target sequence, or may comprise a region of mismatch (i.e., a mismatch motif). The sequence of the ASO can correspond to the full-length target sequence, or a subsequence thereof.

The ASO may reduce or inhibit the expression of a target gene or nucleic acid sequence in a cell via a variety of mechanisms, some of which are described below. In one embodiment, the ASO forms part of a gene editing complex for directing a nuclease to a target site for site-specific cleavage of DNA.

In those embodiments in which the ASO reduces expression of a target gene or sequence by complementary base pairing and degradation of mRNA, the ASO exerts its effects via base-pairing with complementary sequences of a target mRNA and induces mRNA cleavage. The ASO may prevent or reduce the translation of a complementary RNA strand by binding to the RNA. ASOs can be used to target a specific, complementary (coding or non-coding) RNA. If binding occurs, a target sequence can be degraded by the enzyme RNase H, which exists in the nucleus and/or cytoplasm of cells. In one embodiment, the ASO is a “gapmer” sequence that comprises 2-5 chemically modified nucleotides on each terminus blanking a central gap region of DNA (e.g., 8-10 base “gap”). The chemically modified nucleotides decrease degradation by nucleases and increase affinity of the ASO for the target sequence. The gap allows formation of a hybrid sequence that can be cleaved by RNase H. In addition, an oligonucleotide can be chemically modified using known methods to recruit RNase H.

In another embodiment, the ASO may bind a target mRNA and block gene expression. ASOs that function by blocking gene expression are known as “steric blockers” and block binding of the ribosome, thereby preventing or reducing translation of the target nucleotide sequence.

In a further embodiment, the ASO may modulate splicing of a pre-mRNA sequence. ASOs can be designed to target sequences within a pre-mRNA to affect splicing and increase the production of a desired isoform. The ASO can be used, for example, to remove a mutant exon, thereby restoring a proper reading frame and producing a more functional protein product.

In one embodiment, the ASO comprises from about 15 to about 500 nucleotides or from about 20 to about 300 nucleotides or from about 25 to about 200 nucleotides or from about 30 to about 150 nucleotides.

In general, the ASO may comprise a “mismatch motif” or “mismatch region”, which refers to a portion of the ASO sequence that does not have 100% complementarity to its target sequence. An ASO may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

The nucleotides of the ASO may or may not be chemically modified. The modification may improve stability of the ASO, such as increase nuclease resistance in addition to protection provided by the LNP. Moreover, in some examples of the disclosure, the chemical modification improves potency and/or selectivity by increasing binding affinity of the ASO with its complementary sequences. Examples of optional modifications include, but are not limited to, 2′-O-alkyl modifications such as 2′-O-Me or 2′-O-methoxyethyl modifications and 2′-halogen modifications such as 2′-fluoro modifications. In yet further embodiments, the ASO comprises one, two, three, four, or more 2′-deoxy nucleotides, e.g., in the sense and/or antisense strand of the double-stranded region. Alternately or additionally, in some embodiments, the ASO comprises phosphate backbone modifications, such as a phosphorothioate backbone modification. Additional backbone modifications include backbone analogues such as locked nucleic acid (LNA). A non-limiting example is a structure that contains a methylene bridge between the 2′ and 4′ positions of the ribose, which “locks” the ribose ring in a conformation that facilitates binding to a complementary nucleic acid sequence. A related bridge modification is a bridged nucleic acid (BNA). Further examples include ASOs with a peptide backbone (PNA), CpG oligomers, among others known to those of skill the art.

The ASO encapsulated in the LNP is generally single-stranded. However, in some examples of the disclosure, the ASO has self-complementary sequences. In such embodiments, the ASO may form one or more stem loop or hairpin structures within the strand.

In some embodiments, the ASO is covalently bound to one or more other moieties to form a conjugate. In some embodiments, the conjugates are selected based on their ability to facilitate delivery of the ASO to an organism or into cells. An ASO may be bound to a moiety at, for example, the 5′ end of the antisense strand, the 3′ end of the antisense strand, the 5′ end of the sense strand, the 3′ end of the sense strand, or to a nucleotide at a position that is not at the 3′ end or 5′ end of either strand.

Examples of conjugates include but are not limited to one or more of an antibody or fragments thereof, a peptide, an amino acid, an aptamer, a phosphate group, a cholesterol moiety, a lipid, a cell-penetrating peptide, a polymer, such as a hydrophilic polymer, such as polyethylene glycol, and a sugar group, which includes a sugar monomer, an oligosaccharide and modifications thereof. In one non-limiting example, the conjugate is N-Acetylgalactosamine (GalNAc).

Methods of designing antisense oligonucleotides are known in the art and can be readily adapted to produce an antisense oligonucleotide that targets any polynucleotide sequence. Selection of antisense oligonucleotide sequences specific for a given target sequence may be based upon analysis of the chosen target sequence and determination of secondary structure, Tₘ, binding energy, and relative stability. The ASO sequence can be arrived at by computational design or by experimentation.

Improved Biodistribution of Oligo-LNPs with Increasing Neutral Lipid Content

As described in the Example section, the oligo-LNPs of the disclosure having elevated neutral lipid content may provide improved biodistribution to a wider range of tissues and/or organs than an Onpattro-type baseline formulation as described in the Example section herein. In a further embodiment, the baseline formulation may be (a) an otherwise identical LNP having 10 mol % lower levels of the same neutral lipid; (b) when the N/P of the LNP is equal to or greater than 4, an otherwise identical LNP having an N/P that is 1 or 3; and/or (c) when the lipid nanoparticle has a weight nucleic acid/micromole of total lipid that is 0.05:1 to 1:1, an otherwise identical LNP having a weight nucleic acid/micromole of total lipid that is 0.20:1 less than that of the lipid nanoparticle of the disclosure.

The LNP of the disclosure in one embodiment exhibits increased biodistribution in the liver, spleen, bone marrow, heart, lung, kidney, abdominal skin, back skin and/or ear in a specified mouse model than the relevant baseline. In another embodiment, this includes increased biodistribution to extrahepatic tissues selected from the spleen, bone marrow, heart, lung, kidney, abdominal skin, back skin and/or ear relative to the relevant baseline. In another embodiment, this includes increased biodistribution to extrahepatic tissues selected from the spleen or bone marrow relative to the relevant baseline. Whether or not an LNP encapsulating oligonucleotide exhibits such enhanced biodistribution to one or more of a given tissue or organ relative to a baseline oligo-LNP formulation is determined by biodistribution studies in an in vivo mouse model as detailed in the Example section. A fluorescent lipid marker (DiD as described in the Materials and Methods) is used to assess biodistribution of the oligo-LNP in a given tissue or organ relative to the baseline. The animal studies are used as a proxy to assess the biodistribution in a subject, such as a patient in a clinical setting.

In one embodiment, the lipid nanoparticle exhibits at least a 5%, 10%, 20%, 30%, 40%, 50%, 60%, 70%, 80%, 90%, 100%, 110%, 120%, 130%, 140%, 150%, 160%, 170%, 180%, 190% or 200% increase in biodistribution as measured in vivo in the liver, spleen, bone marrow, heart, lung, kidney, abdominal skin, back skin and/or ear of a mouse relative to any one of the above-described relevant baselines, wherein the biodistribution is measured in a mouse model by detection of a lipid marker at 1, 4, 10 and/or 24 hours post-administration. The measurement is carried out on tissue homogenates of one or more of the foregoing tissues or organs as set forth in the Example section.

The percentage increase in fluorescence relative to the relevant baseline is determined by comparing the fluorescence signal of an LNP being assessed in a relevant tissue and/or organ per mg of tissue homogenate to a tissue homogenate fluorescent signal resulting from administration of a baseline LNP.

The oligo-LNPs being compared are prepared using identical materials and methods. In other words, the two formulations compared have the same ionizable lipid, PEG-lipid and sterol and are prepared using rapid ethanol injection as set out in the Materials and Methods.

The biodistribution is assessed at the same time point post-administration (1, 4, 10 and/or 24 hours) to the same mice and using the same analytical technique to measure the marker lipid (see Materials and Methods).

In those embodiments in which the baseline formulation has 10 mol % less neutral lipid (e.g., DSPC or sphingomyelin) than the LNP of the disclosure, the neutral lipid may be decreased in the baseline at the expense of both cholesterol and ionizable lipid in equal proportions but keeping the ionizable lipid:cholesterol (mol:mol) constant between baseline and LNP of the disclosure.

### Clinical and Non-Clinical Uses of the LNP Herein

In some embodiments, the oligo-LNP is part of a pharmaceutical composition and is administered to treat and/or prevent a disease condition. The treatment may provide a prophylactic (preventative), ameliorative or a therapeutic benefit. The pharmaceutical composition will be administered at any suitable dosage.

The oligo-LNPs described herein may be used to treat and/or prevent any disease, disorder or condition in a mammalian subject. This includes a disease, disorder or condition, such as cancer, infectious diseases such as bacterial, viral, fungal or parasitic infections, inflammatory and/or autoimmune disorders, including treatments that induce immune tolerance and cardiovascular diseases such as hypertension, cardiac arrhythmia and restenosis.

Examples of cancers include lung cancer, colon cancer, rectal cancer, anal cancer, bile duct cancer, small intestine cancer, stomach (gastric) cancer, esophageal cancer; gallbladder cancer, liver cancer, pancreatic cancer, appendix cancer, breast cancer, ovarian cancer; cervical cancer, prostate cancer, renal cancer (e.g., renal cell carcinoma), cancer of the central nervous system, glioblastoma, skin cancer, lymphomas, choriocarcinomas, head and neck cancers, osteogenic sarcomas, and blood cancers. Non-limiting examples of specific types of liver cancer include hepatocellular carcinoma (HCC), secondary liver cancer (e.g., caused by metastasis of some other non-liver cancer cell type), and hepatoblastoma.

Non-limiting examples of other diseases, disorders or conditions that may be treated by the oligo-LNPs herein and that may be attributed at least in part to an immunological disorder include colitis, Crohn's disease, allergic encephalitis, allograft transplant/graft vs. host disease (GVHD), diabetes and multiple sclerosis.

The LNPs herein may also be used in other applications besides the treatment and/or prevention of a disease or disorder. The LNPs may be used to treat conditions such as aging, preventative medicine and/or as part of a personalized medicine regime. In further embodiments, the LNP is used in a diagnostic application.

In one embodiment, the LNP is part of a pharmaceutical composition administered parenterally, i.e., intra-arterially, intravenously, subcutaneously or intramuscularly. In yet a further embodiment, the pharmaceutical compositions are for intra-tumoral administration. In another embodiment, the pharmaceutical compositions are administered intranasally, intravitreally, subretinally, intrathecally or via other local routes. In some embodiments, the oligo-LNP is applied or administered to the skin.

The pharmaceutical composition comprises pharmaceutically acceptable salts and/or excipients. As used herein, the term “pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

As used herein, the term “excipient” means the substances used to formulate active pharmaceutical ingredients (API) into pharmaceutical formulations. Non-limiting examples include mannitol, Captisol®, lactose, starch, magnesium stearate, sodium saccharine, talcum, cellulose, sodium crosscarmellose, glucose, gelatin, sucrose, magnesium carbonate, and the like. Acceptable excipients are non-toxic and may be any solid, liquid, semi-solid excipient that is generally available to those of skill in the art.

The compositions described herein may be administered to a subject. The term subject as used herein includes a human or a non-human subject, including a mammal. The oligo-LNP may be administered as part of a preventative treatment and so the subject is not limited to a patient.

The examples are intended to illustrate the preparation of specific lipid nanoparticle oligo preparations and properties thereof but are in no way intended to limit the scope of the invention.

### EXAMPLES

### Materials and Methods

### Preparation of Lipid Nanoparticles Containing Oligonucleotide

The LNPs were prepared by dissolving siRNA firefly luciferase or scrambled antisense oligonucleotide (ASO) in 25 mM sodium acetate, pH 4.0, while the lipid components at the specified mole % were dissolved in absolute ethanol. The lipophilic, fluorescent dye, DiD perchlorate (2-[5-(1,3-Dihydro-3,3-dimethyl-1-octadecyl-2H-indol-2-ylidene)-1,3-pentadien-1-yl]-3,3-dimethyl-1-octadecyl-3H-indolium perchlorate), was included in the LNPs to assess in vivo biodistribution. The lipids in ethanol and the siRNA or ASO in buffer were combined in a 1:3 volume by volume ratio using a t-junction with dual-syringe. The solutions were pushed through the t-junction at a combined flow rate of 20 mL/min (5 mL/minute for the lipid-containing syringe, 15 mL/minute for the siRNA- or ASO-containing syringe). The mixture was subsequently dialyzed overnight against ˜400-1000 volumes of 1× phosphate buffered saline, pH 7.4 using Spectro/Por dialysis membranes (molecular weight cut-off 12 000-14 000 Da) in order to remove ethanol from the formulation.

### Biophysical Characterization

Analysis of lipid nanoparticle size and morphology: LNP size (number weighting) and polydispersity indexes (PDI) was determined by dynamic light scattering (DLS) using a Malvern Zetasizer NanoZS™ particle sizer (Worcestershire, UK).

Analysis of Lipid Nanoparticle siRNA or ASO Encapsulation Efficiency

The siRNA or ASO encapsulation efficiency was determined using the Quant-iT RiboGreen™ RNA assay (Life Technologies™, Burlington, ON). Briefly, LNP-siRNA or LNP-ASO was incubated at 37° C. for 10 min in the presence or absence of 1% Triton X-100 (Sigma-Aldrich™, St. Louis, MO) followed by the addition of the RiboGreen™ reagent. The fluorescence intensity (Ex/Em: 480/520 nm) was determined and samples treated with Triton X-100 represent total siRNA or ASO while untreated samples represent unencapsulated siRNA or ASO.

### Determination of N/P Ratio of Lipid Nanoparticles

The total number of negative charges (P) is first calculated by employing the average molecular weight of a nucleic acid base pair (˜650 g/mol/bp), the size of the nucleic acid construct in number of bp, and the number of negative charges (2/bp). This value in mol*negative charges is multiplied by Avogadro's constant, 6.022E23/mol, resulting in the total number of negative charges.

Likewise, the total number of positive charges on the ionizable lipid can be calculated by first using the molecular weight and the amount of the ionizable lipid to provide the moles of ionizable lipid used in the formulation. This value multiplied by the number of positive charges per molecule (1), provides a value in mol*positive charges which is subsequently multiplied by Avogadro's constant, 6.022E23/mol, resulting in the total number of positive charges. The N/P ratio is calculated by taking the ratio of positive charges on the ionizable lipid to negative charges on the nucleic acid.

### Measurement of Fluorescence in Intact Organs/Tissues In Vivo

The LNPs at an siRNA concentration of 1.0 mg/kg were injected intravenously (i.v.) in CD-1 mice at a volume using the formula weight of the mouse (in grams)*10 μL. At 24 hours post-injection, mice were anesthetized in 5% isofluorane (set to 1% air flow) followed by CO₂ to induce asphyxiation until the animals lost their reflexes. Cardiac puncture was performed using a 1 mL syringe needle and blood was collected. The animals were subsequently imaged on an In Vivo Imaging System (IVIS™) manufactured by PerkinElmer™.

After imaging, animals were cut from the bladder to the rib cage and the skin was pinned back. Spleen (S), lung (Lu), liver (Li) and kidney (K) were removed from the abdominal cavity and placed on a plastic dish. The animals with the organs intact were imaged on the IVIS™ imager as well as the organs subsequently removed from the mice and placed in the plastic dish.

### Tissue Homogenate Assay

Tissues were removed from the mice and placed in 2 mL tubes and snap frozen in liquid nitrogen. The tissues were subsequently frozen at −80° C. An appropriate volume of GLO™ lysis buffer from Promega™ was added to each of the tubes, ensuring that the samples remained frozen before addition of the lysis buffer. Samples were placed in a FastPrep™ homogenizer and the homogenizer was operated and repeated 2 times for a total of three rounds. The homogenized samples were centrifuged at room temperature and subsequently homogenate was added to a black plate. The plate was transferred to a plate reader and the fluorescence was read at 640 nm excitation/720 nm emission.

If bone marrow tissue homogenate is analyzed, femur is removed from the mouse. A cut is made to the femur and it is then centrifuged to isolate the bone marrow. The bone marrow is subsequently treated as described above for the tissues to prepare a homogenate and measure fluorescence.

### Cryo-TEM

LNPs were concentrated to between 15-25 mg/mL estimated total lipid prior to cryo-TEM imaging. A defined volume, for example 2-4 uL, of the resulting LNP solution was applied to a glow-discharged copper grid, and plunge-frozen using an FEI Mark IV Vitrobot to generate vitreous ice. These grids were stored in liquid nitrogen until imaged by an FEI Titan Krios or an FEI Glacios TEM. The instrument was operated at 200 kV in low-dose conditions and the resulting images were obtained using a bottom-mount FEI Falcon direct electron detector camera at 47-88,000× magnification with an under-focus of 0.5-2 μm in order to enhance contrast.

### Example 1: Elevated Levels of Neutral Lipid (e.g., DSPC) in siRNA-LNPs and Reduced Levels of Ionizable Lipid do not Negatively Impact Biophysical Characteristics

A variety of siRNA-LNPs (firefly luciferase) with a range of DSPC contents (10 mol % to 55 mol %) at the expense of ionizable lipid and cholesterol were prepared to assess their biophysical characteristics. In particular, the following four-component siRNA-LNP formulations (reported in mol %) comprising ionizable lipid (MC3 or MF019 as indicated); DSPC; and cholesterol with PEG-lipid (PEG₂₀₀₀-DMG) held constant at 1.5 mol % were evaluated for encapsulation efficiency, size and polydispersity index (PDI) as per the Materials and Methods. The N/P of each LNP was either 3 or 6 as indicated in Table 1.

TABLE 1
Distearoylphosphatidylcholine (DSPC) LNP formulations
tested for biophysical properties

| | Ionizable | DSPC | Chol | PEG-lipid | |
|---|---|---|---|---|---|
| LNP | lipid mol %* | mol % | mol % | mol % | N/P |
| B1 | 50 MC3 | 10 | 38.5 | 1.5 | 3 |
| B2 | 33 MC3 | 40 | 25.5 | 1.5 | 3 |
| B3 | 30.2 MC3 | 45 | 23.3 | 1.5 | 3 |
| B4 | 27.4 MC3 | 50 | 21.1 | 1.5 | 3 |
| B5 | 24.6 MC3 | 55 | 18.9 | 1.5 | 3 |
| B6 | 33 MF019 | 40 | 25.5 | 1.5 | 3 |
| B7 | 30.2 MF019 | 45 | 23.3 | 1.5 | 3 |
| B8 | 27.4 MF019 | 50 | 21.1 | 1.5 | 3 |
| B9 | 24.6 MF019 | 55 | 18.9 | 1.5 | 3 |
| B10 | 33 MF019 | 40 | 25.5 | 1.5 | 6 |
| B11 | 30.2 MF019 | 45 | 23.3 | 1.5 | 6 |
| B12 | 27.4 MF019 | 50 | 21.1 | 1.5 | 6 |
| B13 | 24.6 MF019 | 55 | 18.9 | 1.5 | 6 |
| B14 | 20 MF019 | 40 | 38.5 | 1.5 | 3 |
| B15 | 20 MF019 | 45 | 33.3 | 1.5 | 3 |
| B16 | 20 MF019 | 50 | 28.5 | 1.5 | 3 |
| B17 | 20 MF019 | 55 | 23.5 | 1.5 | 3 |

*MF019 is an ionizable sulfur lipid described in PCT Patent Application No. PCT/CA2022/050042, which is incorporated herein by reference; and MC3 denotes DLin-MC3-DMA ionizable lipid (dilinoleyl-methyl-4-dimethylaminobutyrate).

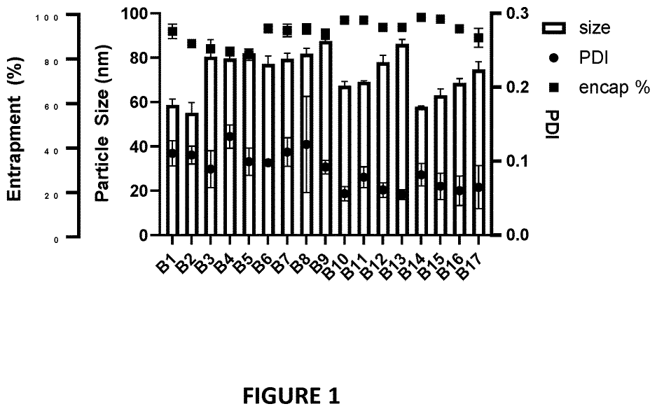

Size, polydispersity (PDI) and encapsulation percent are shown in FIG. 1 for each of the formulations in Table 1 above.

It was found by the inventors that each of the formulations exhibited acceptable biophysical characteristics despite increasing levels of DSPC and decreasing ionizable lipid content. While it would be expected by a person of ordinary skill in the art that as ionizable lipid is decreased, encapsulation efficiency of siRNA would decrease, the results in FIG. 1 surprisingly show that the encapsulation efficiency was relatively constant among the formulations tested even as ionizable lipid content decreased (with a concomitant increase in neutral lipid content).

### Example 2: Elevated Levels of Neutral Lipid (e.g., DSPC) in siRNA-LNPs Significantly Improve In Vivo Biodistribution

The effect of increasing the amount of DSPC from 40 mol % to 50 mol % in a four-component LNP containing siRNA cargo at an N/P of 6 was evaluated in vivo.

Formulations B10, B12 and B6 in Table 1 above were selected for the in vivo biodistribution studies in CD-1 mice. These formulations are summarized in Table 2 below:

TABLE 2
DSPC LNP formulations selected for in vivo biodistribution

| | MF019 | | | |
|---|---|---|---|---|
| | ionizable lipid | | | |
| LNP | mol %* | DSPC mol % | Chol mol % | N/P |
| B10 | 33 | 40 | 25.5 | 6 |
| B12 | 27.4 | 50 | 21.1 | 6 |
| B6 | 33 | 40 | 25.5 | 3 |

*MF019 is an ionizable lipid described in PCT/CA2022/050042 (ibid).

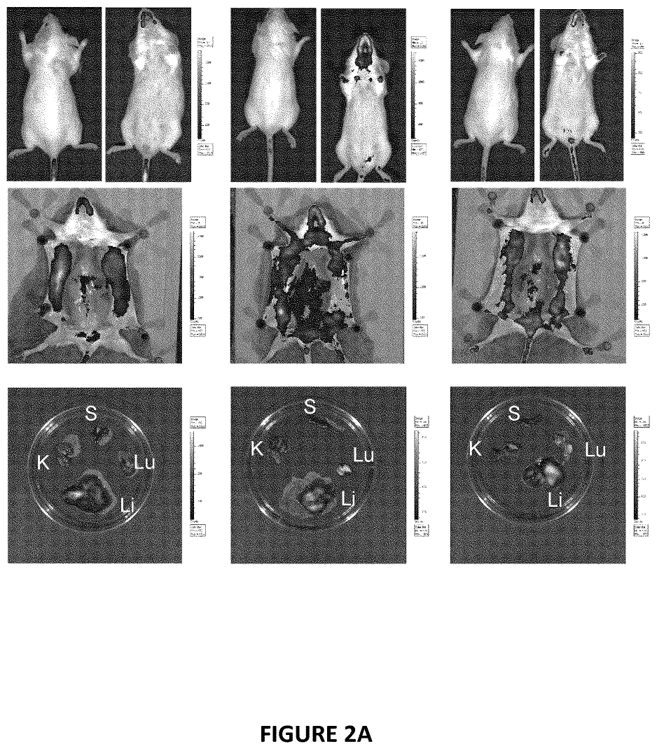

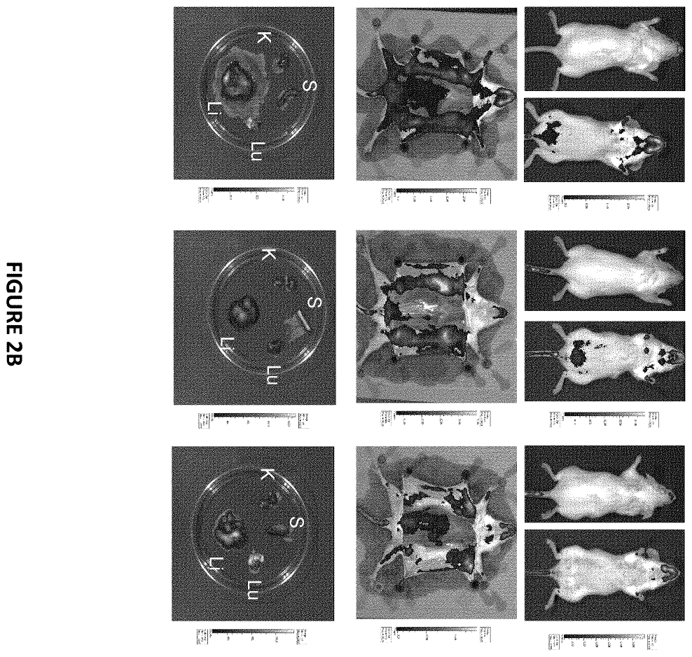

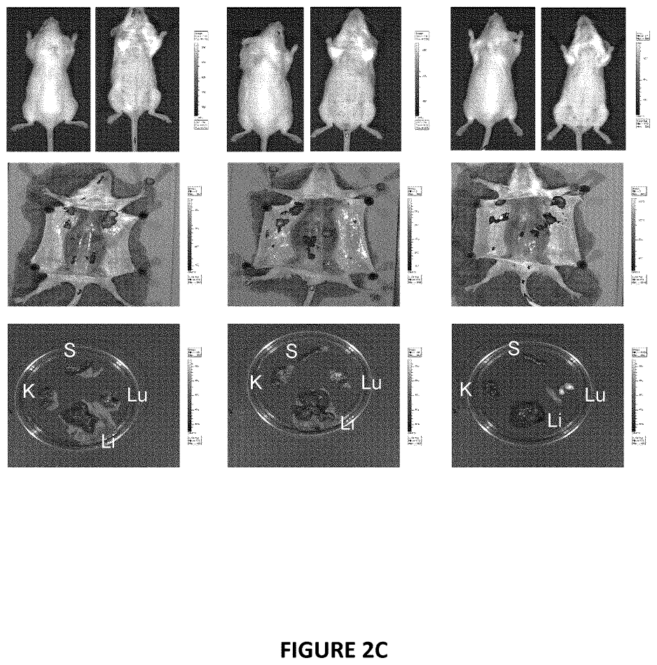

As can be seen in FIGS. 2A, 2B and 2C, the siRNA-LNPs having 50 mol % DSPC showed significant fluorescence intensity in both the liver and spleen as determined by detecting lipophilic fluorescent dye DiD perchlorate 2-[5-(1,3-Dihydro-3,3-dimethyl-1-octadecyl-2H-indol-2-ylidene)-1,3-pentadien-1-yl]-3,3-dimethyl-1-octadecyl-3H-indolium perchlorate at 24 hours post-injection. By contrast, the LNPs having only 40 mol % DSPC exhibited fluorescence in the liver, but a minimal signal in the spleen. In addition, there was almost no detectable fluorescence in the organs examined for LNPs having an N/P of 3 (at 40 mol % DSPC).

### Example 3: Elevated Levels of Sphingomyelin in siRNA-LNPs and Reduced Levels of Ionizable Lipid do not Negatively Impact Biophysical Characteristics

A variety of siRNA-LNPs having a sphingomyelin content ranging from 10 mol % to 55 mol % were prepared to assess their biophysical characteristics. The LNPs also contained varying molar percentages of ionizable lipid and cholesterol (as indicated) with PEG-lipid (PEG₂₀₀₀-DMG) held constant at 1.5 mol %. The siRNA-LNPs were evaluated for encapsulation efficiency, size and polydispersity index (PDI) as set out in the Materials and Methods. The sphingomyelin content was increased at the expense of both cholesterol and ionizable lipid below and the N/P was either 3 or 6 as indicated in Table 3.

TABLE 3
Sphingomyelin LNP formulations tested for biophysical properties

| | Ionizable | SM | Chol | PEG-lipid | |
|---|---|---|---|---|---|
| LNP | lipid mol %* | mol % | mol % | mol % | N/P |
| A1 | 50 MC3 | 10 | 38.5 | 1.5 | 3 |
| A2 | 39.5 MC3 | 40 | 27.6 | 1.5 | 3 |
| A3 | 33 MC3 | 45 | 25.5 | 1.5 | 3 |
| A4 | 30.2 MC3 | 50 | 23.3 | 1.5 | 3 |
| A5 | 27.4 MC3 | 55 | 21.1 | 1.5 | 3 |
| A6 | 24.6 MC3 | 40 | 18.9 | 1.5 | 3 |
| A7 | 35.9 MF019 | 45 | 27.6 | 1.5 | 3 |
| A8 | 33 MF019 | 50 | 25.5 | 1.5 | 3 |
| A9 | 30.2 MF019 | 45 | 23.3 | 1.5 | 3 |
| A10 | 27.4 MF019 | 50 | 21.1 | 1.5 | 3 |
| A11 | 24.6 MF019 | 55 | 18.9 | 1.5 | 3 |
| A12 | 35.9 MF019 | 35 | 27.6 | 1.5 | 6 |
| A13 | 33 MF019 | 40 | 25.5 | 1.5 | 6 |
| A14 | 30.2 MF019 | 45 | 23.3 | 1.5 | 6 |
| A15 | 27.4 MF019 | 50 | 21.1 | 1.5 | 6 |
| A16 | 24.6 MF019 | 55 | 18.9 | 1.5 | 6 |

*MF019 is an ionizable sulfur lipid described in PCT/CA2022/050042 (ibid); and MC3 denotes DLin-MC3-DMA ionizable lipid (dilinoleyl-methyl-4-dimethylaminobutyrate).

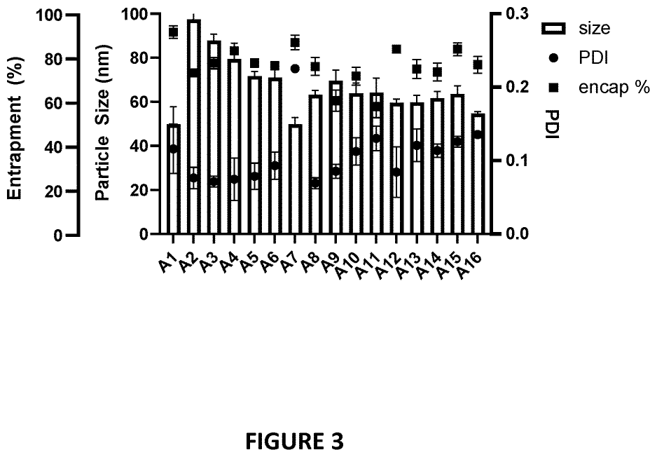

Size, polydispersity (PDI) and encapsulation percent are shown in FIG. 3 for each of the formulations in Table 3 above.

It was found by the inventors that each of the siRNA-LNP formulations generally exhibited acceptable biophysical characteristics despite increasing levels of sphingomyelin and decreasing ionizable lipid content.

### Example 4: Elevated Levels of Neutral Lipid (e.g., DSPC and Sphingomyelin) in siRNA-LNPs Improve Biodistribution In Vivo as Measured in Tissue Homogenates

Biodistribution studies were conducted for various four-component siRNA-LNP formulations containing 40 and 50 mol % DSPC or egg sphingomyelin (ESM). The results show that siRNA-LNPs having 50 mol % DSPC or ESM in most cases had significantly higher concentrations of LNP in the organs measured (tissue homogenate) relative to LNPs having 10 mol % less of the neutral lipid (40 mol %). The neutral lipid contents (DSPC and ESM) were increased at the expense of both ionizable lipid and cholesterol.

The results also show that an siRNA-LNP having an N/P of 3 and 40 mol % DSPC had significantly reduced fluorescence intensity relative to the same formulation having an N/P of 6.

The LNPs that were examined are set out in Table 4 below.

TABLE 4
DSPC and sphingomyelin siRNA-LNP formulations examined
in tissue homogenate murine biodistribution studies

| | | | | PEG-lipid | | |
|---|---|---|---|---|---|---|
| | Ionizable | Mol % of | Chol | mol % | | Encap- |
| | lipid mol | neutral | mol | (PEG2000- | | sulation |
| LNP | % (MF019)* | lipid | % | DMG) | N/P | (%) |
| A | 33 | 40 (ESM) | 25.5 | 1.5 | 6 | 98.3 |
| B | 27.4 | 50 (ESM) | 21.1 | 1.5 | 6 | 98.2 |
| C | 33 | 40 (DSPC) | 25.5 | 1.5 | 6 | 94.1 |
| D | 27.4 | 50 (DSPC) | 21.1 | 1.5 | 6 | 96.5 |
| E | 33 | 40 (DSPC) | 25.5 | 1.5 | 3 | 94.0 |

*MF019 is an ionizable sulfur lipid described in PCT/CA2022/050042 (ibid).

The LNP formulations all exhibited high encapsulation efficiency (>90%) at ionizable lipid concentrations of 27.4 mol % and 33 mol %.

The half-maximal inhibitory concentration (IC₅₀) values for most of the LNPs were within an acceptable range.

Tissue homogenates were prepared as described in the Materials and Methods section. Fluorescence intensity of the tissue homogenates was measured as described using DiD fluorescent marker.

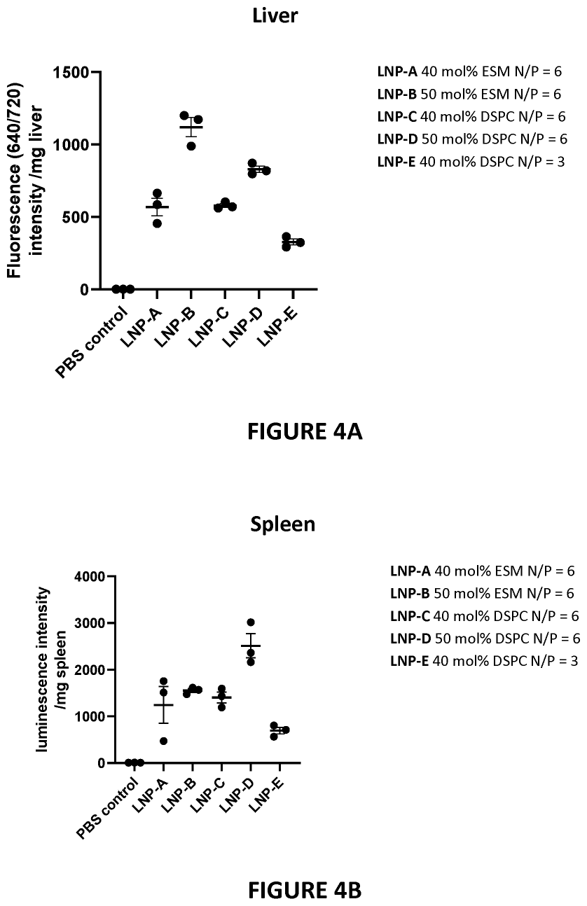

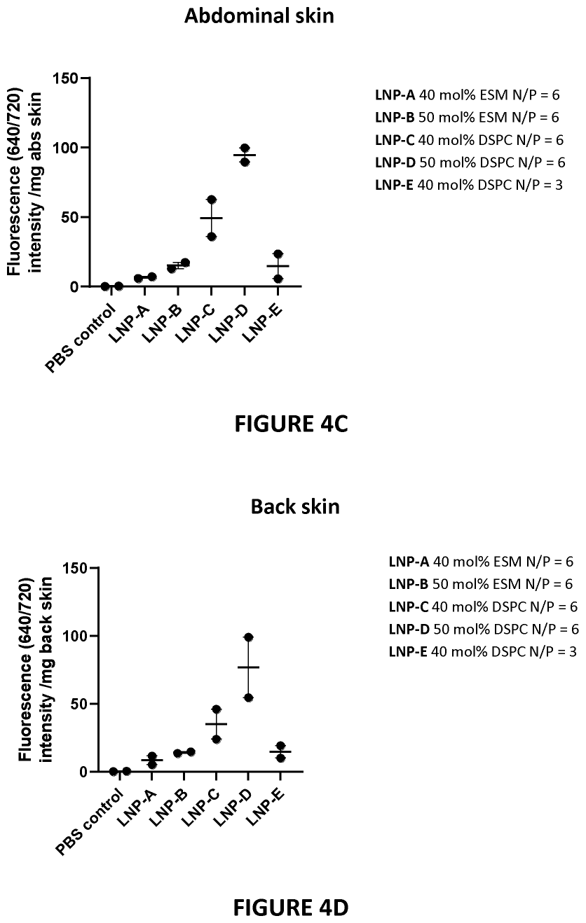

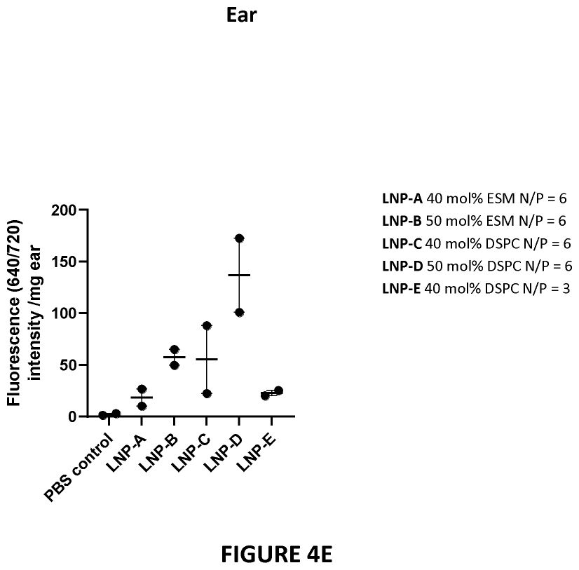

As shown in FIG. 4A, the fluorescence intensity of LNPs containing 50 mol % sphingomyelin in the liver homogenate was increased about 2-fold over that of LNPs containing only 40 mol % sphingomyelin (LNP-A and LNP-B). Similar increases in fluorescence were observed for LNPs containing 50 mol % versus 40 mol % DSPC (LNP-C and LNP-D). The formulations having an N/P of 6 (LNPs A-D) all exhibited increased fluorescent intensities relative to the 40 mol % DSPC LNP (LNP-E) with an N/P of 3.

As shown in FIG. 4B, the fluorescence intensity of LNPs containing 50 mol % sphingomyelin in the spleen homogenate was similar to that of LNPs containing only 40 mol % sphingomyelin (LNP-A and LNP-B). However, a significant increase in fluorescence intensity was observed for LNPs containing 50 mol % DSPC relative to LNPs containing only 40 mol % DSPC (LNP-C and LNP-D). The formulations having an N/P of 6 (LNPs A-D) again all exhibited higher fluorescent intensities relative to the 40 mol % DSPC LNP (LNP-E) having an N/P of only 3.

FIGS. 4C and 4D show biodistribution results for the siRNA-LNPs of Table 4 in the abdominal skin and back skin of mice, respectively. As shown in FIG. 4C, the fluorescence intensity of LNPs containing 50 mol % egg sphingomyelin (ESM) in the abdominal skin was increased over that of LNPs containing only 40 mol % ESM (LNP-A vs LNP-B). A significant increase in fluorescence intensity in abdominal skin was observed for LNPs containing 50 mol % DSPC relative to LNPs containing only 40 mol % DSPC (LNP-C vs LNP-D). All DSPC-containing formulations having an N/P of 6 exhibited higher fluorescence intensities relative to the 40 mol % DSPC formulation having an N/P of only 3. Similar results were observed in the back skin of mice (see FIG. 4D).

Biodistribution results for the ear are shown in FIG. 4E. As shown, the fluorescence intensity of the siRNA-LNPs containing 50 mol % sphingomyelin in the ear was significantly increased over that of LNPs containing only 40 mol % sphingomyelin (LNP-A vs LNP-B). A greater than 2-fold increase in fluorescence intensity in the ear was observed for LNPs containing 50 mol % DSPC relative to LNPs containing only 40 mol % DSPC (LNP-C and LNP-D). Most formulations having an N/P of 6 exhibited a higher fluorescence intensity relative to the 40 mol % DSPC formulation having an N/P of only 3.

### Example 5: Four-Component siRNA-LNPs Having Elevated Levels of Phosphatidylcholine Lipid Exhibit Surprising Morphologies

The morphology of the following LNP formulations were determined by Cryo-TEM as described in the Materials and Methods section. The cargo was siRNA targeting firefly luciferase.

TABLE 5
LNP formulations assessed by cryo-TEM

| | LNP composition: ionizable | | |
|---|---|---|---|
| | lipid/neutral lipid/cholesterol/ | | DSPC |
| Figure | PEG-DMG/DiD (mol/mol) | Molar ratio | content |
| 11A | MF019 ionizable | 33/40/25.5/1.5 | 40 mol % |
| | lipid/DSPC/cholesterol/ | | |
| | PEG₂₀₀₀-DMG | | |
| 11B | MF019 ionizable | 27.4/50/21.1/1.5 | 50 mol % |
| | lipid/DSPC/cholesterol/ | | |
| | PEG₂₀₀₀-DMG | | |
| 11C | MF019 ionizable | 27.4/50/21.1/1.5 | 50 mol % |
| | lipid/ESM/cholesterol/ | | |
| | PEG₂₀₀₀-DMG | | |

The ionizable lipid was MF019 described in PCT/CA2022/050042 filed on Jan. 12, 2022 and published as WO2022/155728A1, which is incorporated herein by reference.

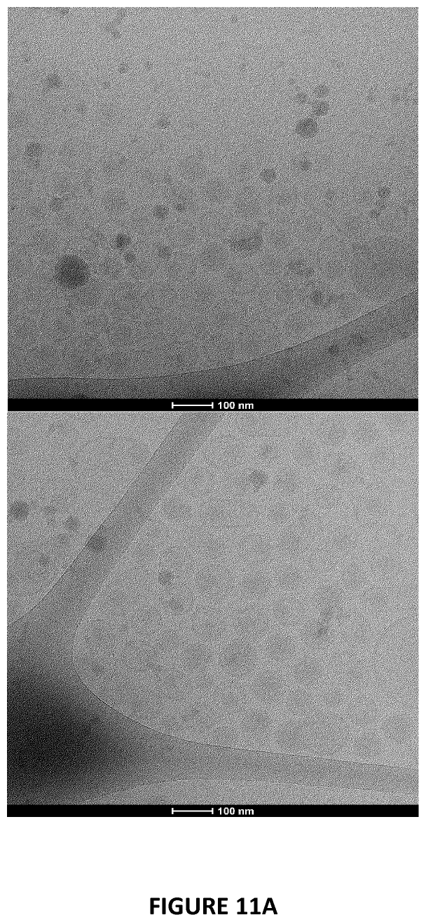

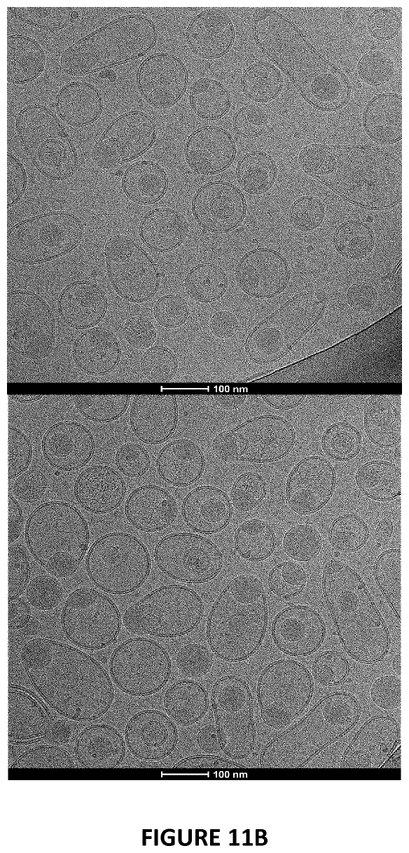

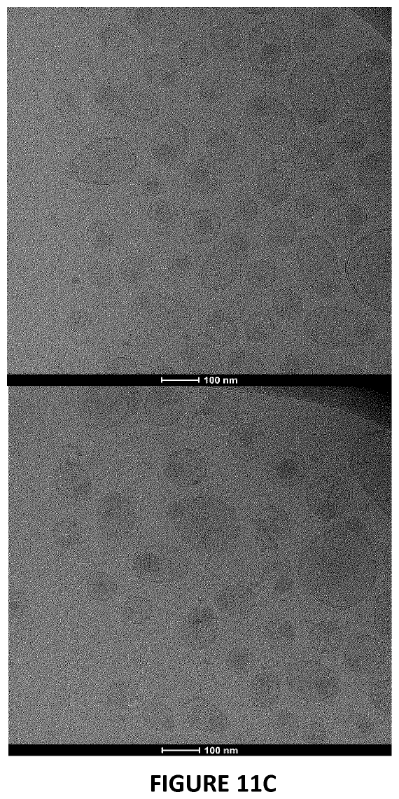

The images in FIGS. 11A, 11B and 11C show that at elevated phosphatidyicholine content (e.g., DSPC), the siRNA-LNPs exhibit a unique morphology with a core having an electron dense region and an aqueous portion, which in turn are surrounded by a bilayer.

### Example 6: Four-Component ASO-LNPs Having Elevated Levels of Phosphatidylcholine Lipid Exhibit Surprising Increases in Extrahepatic Organ Delivery

This example investigates the effect of increasing the content of a representative phosphatidylcholine lipid (DSPC) from 10 mol % to 50 mol % on the biodistribution of an ASO-LNP. The DSPC content was increased at the expense of the ionizable lipid and cholesterol.

Lipid nanoparticles encapsulating 50 mer and 221 mer ssRNA were prepared as described in the method section above. The lipid compositions of the ASO-LNPs investigated are provided in Table 6 below:

TABLE 6
ASO-LNP formulations investigated in biodistribution studies

| | LNP composition: ionizable | | |
|---|---|---|---|
| Formu- | lipid/DSPC/cholesterol/PEG- | DSPC | ssRNA |
| lation | DMG/DiD (mol/mol) | content | cargo size |
| A | 50/10/37.75/1.5/0.75 | 10 mol % | 50-mer |
| | (Onpattro ™)^{a} | | |
| B | 27.4/50/20.35/1.5/0.75^{b} | 50 mol % | 50-mer |
| C | 50/10/37.75/1.5/0.75 | 10 mol % | 221-mer |
| | (Onpattro ™)^{a} | | |
| D | 27.4/50/20.35/1.5/0.75^{b} | 50 mol % | 221-mer |

^{a}The ionizable lipid was MC3 (DLin-MC3-DMA or “MC3” (dilinoleyl-methyl-4-dimethylaminobutyrate))
^{b}The ionizable lipid was MF019 described in PCT/CA2022/050042 filed on Jan. 12, 2022 and published as WO2022/155728A1, incorporated herein by reference.

LNPs A and B encapsulate 50 mer ssRNA and LNPs C and D encapsulate 221 mer ssRNA. LNPs A and C are the Onpattro™ composition with 10 mol % DSPC content while B and D are the compositions with 50 mol % DSPC. The Onpattro™ and high DSPC-containing compositions are respectively 50/10/37.75/1.5/0.75 and 27.4/50/20.35/1.5/0.75 mol/mol of ionizable lipid/DSPC/cholesterol/PEG-DMG/DiD.

The ASOs were non-coding, single-stranded RNAs (ssRNAs) of 50 nucleotides (“50 mer”) and 221 nucleotides (“221 mer”) in length. The ssRNAs were randomly generated such that there would not be any complementary binding for the purposes of the biodistribution experiments. The 50 mer and 221 mer was synthesized by Integrated DNA Technologies™ (IDT). The two sequences are set forth in Table 7 below.

TABLE 7
ASO sequences

| | SEQ | |
|---|---|---|
| ASO | ID | |
| length | NO | Sequence 5′ to 3′ |
| 50 | 1 | 5′-AAAUCAACCCUUACUUGCGGC |
| | | GCUCGGUCUUUACGUUUCUGUUAGUGCCC-3′ |
| 221 | 2 | 5′GCAGUAAUACGACUCACUAUAGGACGGCAACGGUC |
| | | UAUUUGUUCUGCAGGAGUCAAAUAUGACACAGCAGAU |
| | | AUACAGGAAAUCCAAAAAAGCGUUUUAGUGGCUACUA |
| | | AUCACGAAAUCUACCCUUAACUUUACAAGAAUUACAA |
| | | UGCCUCAGGGUCUUUUUUAUCAGUCUUUAAUUGUAUU |
| | | ACCCUGACCCGCCGAUCUACUGGUGGAACCUUCGGAU |
| | | A-3′ |

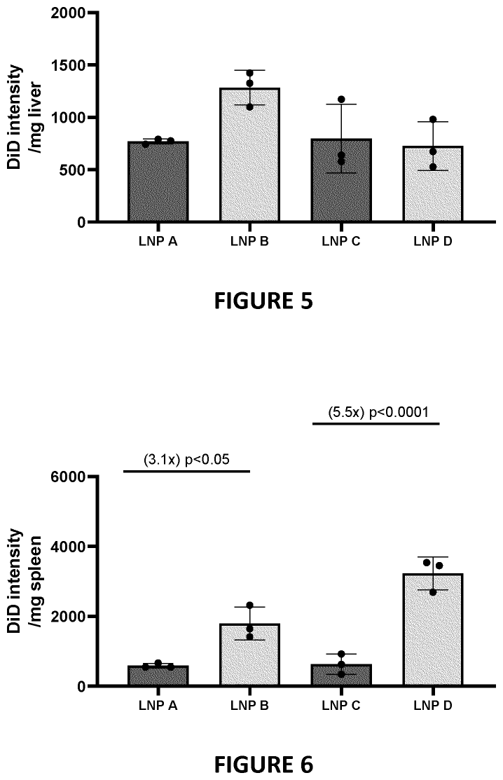

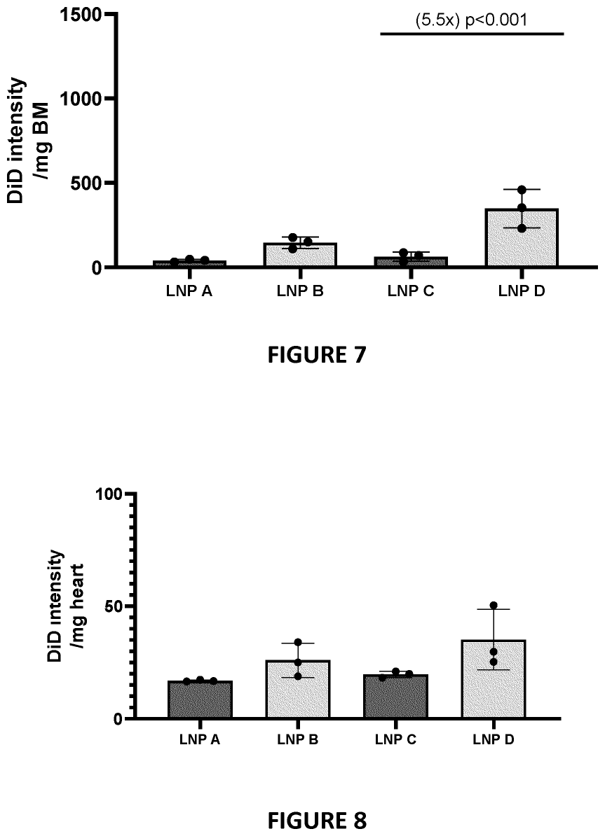

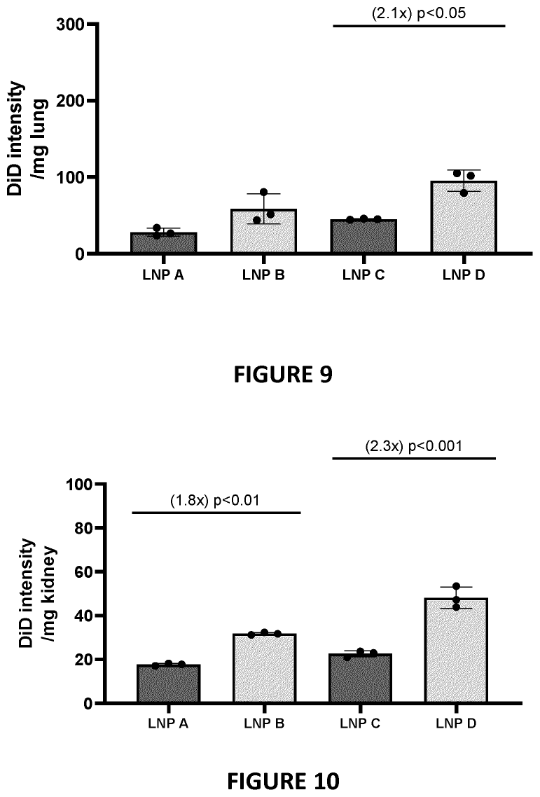

As shown in FIGS. 5-10, the ASO-LNPs comprising 50 mol % DSPC (LNP B and D) exhibited surprising improvements in biodistribution to the spleen, bone marrow, heart, lung and kidney (FIGS. 6-10) relative to the lipid nanoparticles comprising 10 mol % DSPC (LNP A and C) at 24 hours post-administration. The surprising improvements in biodistribution with LNP B and D were observed with both the 50 mer ASO (LNP B) and the 221 mer ASO (LNP D). As shown in FIG. 5, in the liver, the biodistribution of the ASO-LNP having 50 mol % DSPC (LNP B) and the 50 mer ASO was improved over the ASO-LNP having only 10 mol % DSPC (LNP A).

The specification is intended to illustrate embodiments and examples of the invention but is in no way intended to limit the scope of the invention as defined by the appended claims.

1. A lipid nanoparticle comprising:
(i) an oligonucleotide molecule, wherein the oligonucleotide molecule is single-stranded or double-stranded and has a length of between 5 and 500 nucleotides;
(ii) a neutral lipid content of from 30 mol % to 70 mol %;
(iii) an ionizable lipid content of from 5 mol % to 50 mol %;
(iv) a sterol selected from cholesterol or a derivative thereof, and
(v) optionally a hydrophilic polymer-lipid conjugate that is present at a lipid content of 0.5 mol % to 5 mol %,
wherein each lipid content is relative to a total lipid content of the lipid nanoparticle, with the proviso that the phosphatidylcholine lipid content is not egg phosphatidylcholine (EPC).

2. (canceled)

3. A lipid nanoparticle comprising:
(i) an oligonucleotide molecule, wherein the oligonucleotide molecule is single-stranded or double stranded and has a length of between 5 and 500 nucleotides;
(ii) a phosphatidylcholine lipid content of from 30 mol % to 70 mol %;
(iii) an ionizable lipid content of from 5 mol % to 50 mol %;
(iv) a sterol selected from cholesterol or a derivative thereof, and
(v) a hydrophilic polymer-lipid conjugate that is present at a lipid content of 0 mol % to 5 mol %,
wherein each lipid content is relative to a total lipid content of the lipid nanoparticle,
and wherein the lipid nanoparticle comprises a non-homogeneous core, the core having an electron dense region and an aqueous portion, and is surrounded by an external lipid layer, as visualized by cryo-EM.

4. The lipid nanoparticle of claim 1, wherein the neutral lipid is a phosphatidylcholine lipid selected from distearoylphosphatidylcholine (DSPC), dioleoylphosphatidylcholine (DOPC), 1-palmitoyl-2-oleoyl-phosphatidylcholine (POPC), dimyristoyl-phosphatidylcholine (DMPC) or dipalmitoyl-phosphatidylcholine (DPPC).

5. The lipid nanoparticle of claim 4, wherein the phosphatidylcholine lipid is distearoylphosphatidylcholine (DSPC) or dioleoylphosphatidylcholine (DOPC).

6. The lipid nanoparticle of claim 1, wherein the neutral lipid content is between 40 mol % and 60 mol %.

7. The lipid nanoparticle of claim 6, wherein the neutral lipid content is between 42 mol % and 60 mol %.

8. (canceled)

9. (canceled)

10. (canceled)

11. The lipid nanoparticle of claim 1, wherein the ionizable lipid is an amino lipid.

12. The lipid nanoparticle of claim 1, wherein the ionizable lipid is present at less than 40 mol %.

13. The lipid nanoparticle of any claim 1, wherein the hydrophilic polymer-lipid conjugate is a polyethyleneglycol-lipid conjugate.

14. The lipid nanoparticle of claim 1, wherein the sterol is present at from 15 mol % to 45 mol % based on the total lipid present in the lipid nanoparticle.

15. The lipid nanoparticle of claim 1, wherein the sterol is present at from 18 mol % to 40 mol % based on the total lipid present in the lipid nanoparticle.

16. The lipid nanoparticle of claim 1, wherein the lipid nanoparticle exhibits at least a 10% increase in biodistribution in the spleen, bone marrow, heart, lungs, kidney, abdominal skin, back skin and/or ear as compared to a baseline Onpattro-type formulation of MC3/DSPC/cholesterol/PEG-lipid at 50/10/38.5/1.5, mol:mol encapsulating the oligonucleotide molecule, but otherwise measured under an identical set of conditions, and wherein the biodistribution is quantified in an animal model by detection of labelled lipid at 24 hours post-administration.

17. The lipid nanoparticle of claim 1, wherein the oligonucleotide is an antisense oligonucleotide that is single-stranded and has a length of between 30 and 300 nucleotides.

18. The lipid nanoparticle of claim 1, wherein the oligonucleotide is an siRNA.

19. A method for in vivo delivery of an oligonucleotide molecule to a mammalian subject, wherein the oligonucleotide molecule is single-stranded or double-stranded and has a length of between 5 and 500 nucleotides, the method comprising: administering to the mammalian subject the lipid nanoparticle of claim 1.

20. A method for delivering an oligonucleotide molecule to a cell, the method comprising contacting the lipid nanoparticle of claim 1 with the cell in vivo or in vitro.

21. The method of claim 19, wherein the oligonucleotide is an antisense oligonucleotide molecule and accumulates in the spleen, bone marrow, heart, lungs and/or kidney of the mammalian subject at least one day post-administration.

22. The method of claim 19, wherein the lipid nanoparticle is used to treat a disease or disorder that is an autoimmune disorder.

23. The method of claim 19, wherein the lipid nanoparticle is used to treat a disease or disorder that is an infectious disease.

24. The method of claim 19, wherein the lipid nanoparticle is used to treat a disease or disorder that is cancer.

25. (canceled)

26. (canceled)
